(19) United States
(12) Patent Application Publication
(10) Pub. No.: US 2015/0112335 A1
(43) Pub. Date: Apr. 23, 2015

(54) ELECTROSURGICAL DEVICES WITH FLUID FLOW CONTROL

(71) Applicant: Ethicon Endo-Surgery, Inc., Cincinnati, OH (US)

(72) Inventors: Chad P. Boudreaux, Cincinnati, OH (US); Matthew C. Miller, Cincinnati, OH (US); Cory G. Kimball, Cincinnati, OH (US)

(73) Assignee: Ethicon Endo-Surgery, Inc., Cincinnati, OH (US)

(21) Appl. No.: 14/057,682

(22) Filed: Oct. 18, 2013

(51) Int. Cl. A61B 18/14

(52) U.S. Cl. CPC A61B 18/14; A61B 2018/0091
USPC 606/49

(57) ABSTRACT

The disclosure provides various electrosurgical devices comprising a handle assembly comprising a valve having an input port and an output port. The input port is fluidically coupled to either an irrigation source or a suction source. At least one button is operatively coupled to the valve to control flow through the valve. At least one switch is provided to electrically couple energy from an energy source. A trigger and a shaft comprising a slidable element is operatively coupled to the trigger. An electrode is electrically coupled to the switch. The trigger is operable to position the slidable element relative to the electrode to conceal or expose the electrode. The handle assembly may comprise an articulating joint. The handle assembly may comprise a trigger lockout mechanism.

### BACKGROUND

The present disclosure is related generally to electrosurgical devices with various mechanisms for controlling fluid flow. In particular, the present disclosure is related to electrosurgical devices with various mechanisms for controlling fluid flow, such as, irrigation and suction fluid flow, for example. More particularly, the present disclosure is related to electrosurgical devices with articulating manifolds and various mechanisms for controlling fluid flow, such as irrigation and suction fluid flow, for example.

While several devices have been made and used, it is believed that no one prior to the inventors has made or used the device described in the appended claims.

### SUMMARY

In one embodiment, an electrosurgical device comprises a handle assembly comprising: a valve having an input port and an output port, the input port fluidically coupled to either an irrigation source or a suction source; at least one button operatively coupled to the valve to control flow through the valve; at least one switch to electrically couple energy from an energy source; and a trigger; a shaft comprising: a slidable element operatively coupled to the trigger; and an electrode electrically coupled to the switch; wherein the trigger is operable to position the slidable element relative to the electrode to conceal or expose the electrode.

In another embodiment, an electrosurgical device comprises a handle assembly defining a rigid wall; a cam arm supported by the handle assembly and pivotally movable about a first pivot on one side and comprising a roller supported on another side, the cam arm defining a slot therebetween; a first button supported by the handle assembly and pivotally movable about a second pivot, the first button comprising: an arm; and a projecting tab slidably engaged with the slot; a second button pivotally coupled to the cam arm at the first pivot; and a bias element acting on the arm of the first button to force the cam arm to pivotally move in a first direction and the roller to move toward the rigid wall of the handle assembly; wherein pressing the first button overcomes the force of the bias element and the projecting tab applies a force on the slot to pivotally move the cam arm in a second direction and to move the roller away from the rigid wall of the handle assembly.

In yet another embodiment, an electrosurgical device comprises a handle assembly comprising an articulating handle comprising a proximal housing and a distal housing rotatably coupled at an articulation joint; a sealed fluid flow manifold assembly configured to articulate about the articulation joint; and a locking mechanism positioned at the articulation joint to lock the proximal housing and the distal hosing in a configuration.

In addition to the foregoing, various other method and/or system and/or aspects are set forth and described in the teachings such as text (e.g., claims and/or detailed description) and/or drawings of the present disclosure.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

The foregoing summary is illustrative only and is not intended to be in any way limiting. In addition to the illustrative aspects, embodiments, and features described above, further aspects, embodiments, and features will become apparent by reference to the drawings and the following detailed description.

### FIGURES

The novel features of the embodiments described herein are set forth with particularity in the appended claims. The embodiments, however, both as to organization and methods of operation may be better understood by reference to the following description, taken in conjunction with the accompanying drawings as follows.

FIG. 1 depicts a perspective view of an electrosurgical device comprising a handle assembly, a shaft portion, and an electrode portion, the electrosurgical device comprising fluid flow features and a slidably movable element to control exposure of the electrode portion of the electrosurgical device, according to one embodiment.

FIG. 2 depicts a perspective view of the handle assembly of the electrosurgical device shown in FIG. 1, according to one embodiment.

FIG. 3 depicts a perspective view of the shaft portion of the electrosurgical device shown in FIG. 1 configured to couple to the handle assembly of the electrosurgical device shown in FIG. 2, according to one embodiment.

FIG. 4 depicts a side elevational view of the handle assembly of the electrosurgical device shown in FIG. 1 with a trigger portion in a second position and the slidably movable element is in a retracted position, according to one embodiment.

FIG. 5 depicts a distal end view of the shaft portion of the electrosurgical device shown in FIG. 1 that is coupled to the handle assembly of the electrosurgical device shown in FIG. 4, with a slidably movable sheath in the retracted to expose the electrode, according to one embodiment.

FIG. 6 depicts a side elevational view of the handle assembly of the electrosurgical device shown in FIG. 1 where the trigger is in a first position and the slidably movable element is in an extended position to conceal the electrode, according to one embodiment.

FIG. 7 depicts a distal end view of the shaft portion of the electrosurgical device shown in FIG. 1 that is coupled to the handle assembly of the electrosurgical device shown in FIG. 6, with a slidably movable sheath coupled to the slidably movable element in the retracted to expose the electrode portion of the electrosurgical element, according to one embodiment.

FIG. 8 depicts an exploded view of the handle assembly of the electrosurgical device shown in FIG. 2, according to one embodiment.

FIG. 9 depicts a partial cut-away transparent side elevational view of the handle assembly shown in FIG. 2 with the fluid flow control buttons pushed out to disable flow, according to one embodiment.

FIG. 10 depicts a partial cut-away transparent side elevational view of the handle assembly shown in FIG. 2 with the fluid flow control buttons pushed in to enable fluid flow, according to one embodiment.

FIG. 11 depicts a partial cut-away transparent side elevational view of the handle assembly shown in FIG. 2 with the reversing arm operatively coupled to the trigger where the trigger is located in a forward (distal) position as biased by a torsion spring to extend the slide element forward in a distal direction to conceal the electrode, according to one embodiment.

FIG. 12 depicts a partial cut-away transparent side elevational view of the handle assembly shown in FIG. 2 with the trigger in a backward (proximal) position squeezed to overcome the bias force of the torsion spring to retract the slide element backward in a proximal direction to expose the electrode, according to one embodiment.

FIG. 13 depicts a transverse sectional view of the handle assembly shown in FIG. 2 to further illustrate the operation of the fluid flow valves, according to one embodiment.

FIG. 14 depicts a longitudinal sectional view of the handle assembly shown in FIG. 2 to further illustrate the operation of the fluid flow valves, according to one embodiment.

FIG. 15 depicts a partial cut-away transparent view of the handle assembly shown in FIG. 2 to illustrate electrical wiring connections between an energy cable, an energy switch circuit board of the energy switch, and an electrical contact spring, according to one embodiment.

FIG. 16 depicts a right side perspective view of the handle assembly shown in FIG. 15 to illustrate the wiring between the energy cable and the circuit board of the energy switch, according to one embodiment.

FIG. 17 depicts an exploded view of a valve manifold assembly, according to one embodiment.

FIG. 18 depicts a perspective view of an assembled valve manifold assembly shown in FIG. 17, according to one embodiment.

FIG. 19 depicts a button/spring assembly to operate either of fluid flow functions such as irrigation and/or suction, according to one embodiment.

FIG. 20 depicts an exploded view of a “valve manifold assembly/buttons/reversing arm” assembly, according to one embodiment.

FIG. 21 depicts a perspective view of the “valve manifold assembly/buttons/reversing arm” assembly shown in FIG. 20, according to one embodiment.

FIG. 22 depicts a perspective view of the right shroud portion of the handle assembly shown in FIG. 2 with fluid flow hoses threaded through a bottom portion thereof, according to one embodiment.

FIG. 23 depicts a perspective view of the right shroud portion of the handle assembly shown in FIG. 2 with the “valve manifold assembly/buttons/reversing arm” assembly shown in FIG. 20-22 in the process of being attached to the fluid flow hoses, according to one embodiment.

FIG. 24 depicts a side elevational view of the right shroud portion of the handle assembly shown in FIG. 2 with the “valve manifold assembly/buttons/reversing arm” assembly shown in FIG. 23 attached to the fluid flow hoses, according to one embodiment.

FIG. 25 depicts a perspective view of the right shroud portion of the handle assembly shown in FIG. 2 with the “valve manifold assembly/buttons/reversing arm” assembly shown in FIG. 24 attached to the fluid flow hoses and an energy switch circuit board, cable, and retainer/electrical spring contact mounted to the right shroud, according to one embodiment.

FIG. 26 depicts a partial side elevational view of the right shroud portion of the handle assembly shown in FIG. 2 with a reverse arm rotated forward in a maximum distal direction and a slide element slidably attached over a nozzle, according to one embodiment.

FIG. 27 depicts a partial side elevational view of the right shroud portion of the handle assembly shown in FIG. 26 with the reverse arm rotated backward in a maximum proximal direction to lock the slide element in place, according to one embodiment.

FIG. 28 depicts a perspective view of the right shroud portion of the handle assembly shown in FIG. 27 with a trigger attached to thereto and operatively coupled to the reverse arm and a compression spring, according to one embodiment.

FIG. 29 depicts a partial perspective view of the right shroud portion of the handle assembly shown in FIG. 28 with an energy switch and shaft unlock button attached thereto, according to one embodiment.

FIG. 30 depicts a perspective view of a left shroud portion of the handle assembly shown in FIG. 29 in the process of being attached to the right shroud portion of the handle assembly, according to one embodiment.

FIG. 31 depicts a perspective view of an assembled handle assembly shown in FIG. 2, according to one embodiment.

FIG. 32 depicts a transparent side elevational view of a handle assembly of an electrosurgical device comprising a trigger operatively coupled to a slide element by way of a cam arm where the trigger is extended in the distal direction and the slide element is retracted in the proximal direction, according to one embodiment.

FIG. 33 depicts a transparent side elevational view of the handle assembly of an electrosurgical device shown in FIG. 32 with a trigger operatively coupled to a slide element by way of a cam arm where the trigger is retracted in the proximal direction and the slide element is extended in the distal direction, according to one embodiment.

FIG. 34 depicts a perspective transparent view of the handle assembly of the electrosurgical device shown in FIG. 33 with a trigger operatively coupled to a slide element by way of a cam arm where the trigger is retracted in the proximal direction and the slide element is extended in the distal direction with a slide knob element removed to show a shaft locking element, according to one embodiment.

FIG. 35 depicts a transparent side elevational view of a handle assembly of an electrosurgical device comprising a trigger operatively coupled to a slide element by way of a gear train attached to a rack where the trigger is extended in the distal direction and the slide element is retracted in the proximal direction, according to one embodiment.

FIG. 36 depicts a transparent side elevational view of a handle assembly of an electrosurgical device comprising a trigger operatively coupled to a slide element by way of a cable pull system where the trigger is extended in the distal direction and the slide element is retracted in the proximal direction, according to one embodiment.

FIG. 37 depicts a perspective view of a shaft portion of the hand assemblies shown in FIGS. 32-36 coupled to a rotatable knob element that is coupled to a slide element, according to one embodiment.

FIG. 38 depicts a perspective view of rotatable knob element showing a shaft to slide element locking feature and a nozzle portion, according to one embodiment.

FIG. 39 depicts a transparent side elevational view of a handle assembly of an electrosurgical device comprising a tube pinching mechanism engaged with a tube to deactivate fluid flow or suction, according to one embodiment.

FIG. 40 depicts a detailed transparent side elevational view of the handle assembly shown in FIG. 39 with the tube pinching mechanism disengaged with the tube to activate fluid flow or suction, according to one embodiment.

FIG. 41 depicts a transparent perspective view of the handle assembly shown in FIG. 39 with the tube pinching mechanism engaged to deactivate fluid flow or suction, according to one embodiment.

FIG. 42 depicts a transparent perspective view of the handle assembly shown in FIG. 40 with the tube pinching mechanism disengaged with the tube to activate fluid flow or suction, according to one embodiment.

FIG. 43 depicts a side elevational view of a handle assembly of an electrosurgical device comprising an articulating sealed fluid flow manifold assembly with a locking mechanism positioned in a straight/non-rotated (pencil) configuration, according to one embodiment.

FIG. 44 depicts a side elevational view of the handle assembly shown in FIG. 43 arranged in a bent/rotated (pistol) configuration, according to one embodiment.

FIG. 45 depicts a perspective view of the handle assembly shown in FIGS. 43-44 arranged in a straight non-rotated (pencil) configuration, according to one embodiment.

FIG. 46 depicts an exploded view of the handle assembly shown in FIGS. 43-45, according to one embodiment.

FIGS. 47A-D depict a sequence of steps for rotating and locking the handle assembly shown in FIGS. 43-46, according to one embodiment.

FIG. 48 depicts a partial sectional view of the handle assembly shown in FIGS. 43-46 arranged in a flow blocked straight non-rotated (pencil) configuration, according to one embodiment.

FIG. 49 depicts a partial sectional view of the handle assembly shown in FIGS. 43-46 arranged in a flow opened straight non-rotated (pencil) configuration, according to one embodiment.

FIG. 50 depicts a partial sectional view of the handle assembly shown in FIGS. 43-46 arranged in a flow blocked bent rotated (pistol) configuration, according to one embodiment.

FIG. 51 depicts an exploded view of the articulating sealed fluid flow manifold assembly for the handle assembly shown in FIGS. 43-46 showing the flow paths, stop cock valves, and O-ring seal between articulating manifold ends, according to one embodiment.

FIG. 52 depicts a perspective sectional view of the articulating sealed fluid flow manifold assembly for the handle assembly shown in FIGS. 43-46 showing the stop cock valves is a closed position to stop the fluid flow in the flow paths of the articulating manifold, according to one embodiment.

FIG. 53 depicts a sectional view of the articulating sealed fluid flow manifold assembly for the handle assembly shown in FIGS. 43-46 showing one of the flow paths before the closed stop clock valve, according to one embodiment.

FIG. 54 depicts a sectional view of the articulating sealed fluid flow manifold assembly for the handle assembly shown in FIGS. 43-46 showing an electrical wire path, according to one embodiment.

FIG. 55 depicts a sectional view of the articulating sealed fluid flow manifold assembly for the handle assembly shown in FIG. 54 arranged in a straight non-rotated (pencil) configuration showing an electrical wire path arranged, according to one embodiment.

FIG. 56 depicts a sectional view of the articulating sealed fluid flow manifold assembly for the handle assembly shown in FIG. 54 arranged in a bent rotated (pistol) configuration showing an electrical wire path, according to one embodiment.

FIG. 57 depicts a side elevational view of the handle assembly shown in FIGS. 43-46 arranged in a bent rotated (pistol) configuration showing the distance between a distal button and a rotatable knob, according to one embodiment.

FIG. 58 is a perspective view of the handle assembly shown in FIG. 57 arranged in a bent rotated (pistol) configuration showing a distance between a distal button and a rotatable knob, according to one embodiment.

FIG. 59 depicts a side elevational view of the handle assembly shown in FIGS. 43-46 arranged in a straight non-rotated (pencil) configuration showing the distance between a distal button and a rotatable knob, according to one embodiment.

FIG. 60 depicts a diagram for assembling the fluid flow tubes to flow connection components parts of the manifold subassembly for the handle assembly shown in FIGS. 43-46, according to one embodiment.

FIG. 61 depicts an assembly diagram for an articulating manifold subassembly of the handle assembly shown in FIGS. 43-46, according to one embodiment.

FIG. 62 depicts a perspective view of the assembled articulating manifold subassembly of the handle assembly shown in FIG. 61, according to one embodiment.

FIG. 63 depicts an exploded view of a manifold subassembly of the handle assembly shown in FIGS. 43-46, according to one embodiment.

FIG. 64 depicts a perspective view of the assembled manifold subassembly shown in FIG. 63, according to one embodiment.

FIG. 65 depicts a perspective view of the manifold subassembly shown in FIG. 64 with fluid flow control buttons and springs inserted into corresponding slots on the valve manifold, according to one embodiment.

FIG. 66 depicts a perspective view of the manifold subassembly with fluid flcow control buttons and springs coupled thereto as shown in FIG. 65 with circuit board and contact spring attached thereto, according to one embodiment.

FIG. 67 depicts a right proximal portion of a handle assembly for the handle assembly shown in FIGS. 43-46 with an articulation lock and spring inserted in a distal portion thereof, according to one embodiment.

FIG. 68 depicts the right proximal portion of the handle assembly with the articulation lock and spring shown in FIG. 67 coupled to a right distal handle assembly, according to one embodiment.

FIG. 69 depicts the right proximal and distal portions of the handle assembly shown in FIG. 68 with the manifold subassembly shown in FIG. 66 coupled to thereto, according to one embodiment.

FIG. 70 depicts the distal and proximal portions of the handle assembly shown in FIG. 69 with an unlock arm inserted in the right distal handle assembly, according to one embodiment.

FIG. 71 depicts the distal and proximal right handle assembly of the handle assembly shown in FIG. 70 with a left distal portion of the handle coupled thereto and a distal tip coupled to the distal handle assembly, according to one embodiment.

FIG. 72 depicts a left proximal handle assembly coupled to the right handle assembly of the handle assembly shown in FIG. 71, according to one embodiment.

FIG. 73 depicts an exploded view of the handle assembly of the handle assembly shown in FIG. 72 and energy buttons, according to one embodiment.

FIG. 74 depicts a complete handle assembly, according to one embodiment.

FIG. 75 depicts a perspective view of a handle assembly with a trigger lockout mechanism, according to one embodiment, the handle assembly being compatible with the electrosurgical device shown in FIG. 1, according to one embodiment.

FIG. 76 depicts an exploded view of the handle assembly shown in FIG. 75, according to one embodiment.

FIG. 77 depicts a partial transparent side elevational view of the handle assembly shown in FIG. 75 with a trigger portion in a second position and a slidably movable element is in a retracted position, according to one embodiment.

FIG. 78 depicts a partial transparent side elevational view of the handle assembly shown in FIG. 75 with a trigger portion in a first position and a slidably movable element is in an extended position, according to one embodiment.

FIG. 79 depicts a partial transparent side elevational view of the handle assembly shown in FIG. 75 with the trigger portion in a second position and the shaft in a retracted position and fluid flow control buttons pushed out to disable fluid flow, according to one embodiment.

FIG. 80 depicts a partial transparent side elevational view of the handle assembly shown in FIG. 75 with the trigger portion in a first position and the shaft in an extended position and the fluid flow control buttons pushed in to enable fluid flow, according to one embodiment.

FIG. 81 depicts a partial cut-away transparent side elevational view of the handle assembly shown in FIG. 75 with the reversing arm operatively coupled to the trigger where the trigger is located in a forward (distal) position as biased by a torsion spring to extend the slide element forward in a distal direction to conceal the electrode, according to one embodiment.

FIG. 82 depicts a partial cut-away transparent side elevational view of the handle assembly shown in FIG. 75 with the trigger in a backward (proximal) position squeezed to overcome the bias force of the torsion spring to retract the slide element backward in a proximal direction to expose the electrode, according to one embodiment.

FIG. 83 depicts a partial cut-away transparent side elevational view of the handle assembly shown in FIG. 75 with a wire cover, according to one embodiment.

FIG. 84 depicts a partial perspective view of the handle assembly shown in FIG. 75 showing the trigger and reverse arm elements, according to one embodiment.

FIG. 85 depicts a perspective view of the trigger and trigger plate elements of the handle assembly shown in FIG. 75, according to one embodiment.

FIG. 86 depicts a perspective view of a trigger lockout assembly for the handle assembly shown in FIG. 75, according to one embodiment.

FIG. 87 depicts perspective view of an interior portion of a left shroud of the handle assembly shown in FIG. 75 showing the trigger lockout assembly located therein, according to one embodiment.

FIG. 88 depicts a perspective view of a housing portion of the trigger lockout assembly shown in FIG. 86, according to one embodiment.

FIG. 89 depicts a perspective view of the trigger lockout assembly shown in FIG. 86, according to embodiment.

FIG. 90 depicts a path of the pin portion of the trigger lockout assembly through a trigger lockout cam to lock and unlock the trigger, according to one embodiment.

FIG. 91 depicts the trigger lockout assembly shown in FIG. 86 being bypassed by sliding a trigger lockout button, according to one embodiment.

### DESCRIPTION

In the following detailed description, reference is made to the accompanying drawings, which form a part hereof. In the drawings, similar symbols and reference characters typically identify similar components throughout the several views, unless context dictates otherwise. The illustrative embodiments described in the detailed description, drawings, and claims are not meant to be limiting. Other embodiments may be utilized, and other changes may be made, without departing from the scope of the subject matter presented here.

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Before explaining the various embodiments of the surgical devices with close quarter articulation features in detail, it should be noted that the various embodiments disclosed herein are not limited in their application or use to the details of construction and arrangement of parts illustrated in the accompanying drawings and description. Rather, the disclosed embodiments may be positioned or incorporated in other embodiments, variations and modifications thereof, and may be practiced or carried out in various ways. Accordingly, embodiments of the surgical devices with close quarter articulation features disclosed herein are illustrative in nature and are not meant to limit the scope or application thereof. Furthermore, unless otherwise indicated, the terms and expressions employed herein have been chosen for the purpose of describing the embodiments for the convenience of the reader and are not to limit the scope thereof. In addition, it should be understood that any one or more of the disclosed embodiments, expressions of embodiments, and/or examples thereof, can be combined with any one or more of the other disclosed embodiments, expressions of embodiments, and/or examples thereof, without limitation.

Also, in the following description, it is to be understood that terms such as front, back, inside, outside, top, bottom and the like are words of convenience and are not to be construed as limiting terms. Terminology used herein is not meant to be limiting insofar as devices described herein, or portions thereof, may be attached or utilized in other orientations. The various embodiments will be described in more detail with reference to the drawings.

The embodiments described herein provide electrosurgical devices with electrical energy driven tissue sealing (e.g., cauterization) or treatment (e.g., non-reversible electroporation) functionality as well as irrigation and suction functionalities. The electrosurgical devices include handle assemblies that are ergonomically easy to use for the operator (e.g., surgeon). The handle assemblies include suction and irrigation buttons that are easy to press, shaft knobs that are easy to rotate, and energy buttons that are easy to activate making it easy for the device to switch between multiple modes of operation. The suction and irrigation buttons, shaft knobs, and energy activation buttons according to the disclosed embodiments can be manipulated with reduced force and can be operated with one hand to control irrigation and suction, manipulate and rotate the shaft knob, and activate energy.

In various embodiments, electrosurgical devices are provided that can be operated without requiring the operator to manually push/pull the irrigation and suction nozzle or sheath forward/backward to conceal/expose a distal electrode tip during irrigation and suction/cauterization processes. Pushing the nozzle forward or pulling it back is not ergonomic and does not allow the operator to have fine control over the extension of the shaft. Accordingly, various mechanisms are disclosed to improve the ergonomics functionality of such electrosurgical instruments to allow for better shaft extension/retraction, irrigation and suction, and energy activation control.

Accordingly, the various embodiments described herein provide for single hand use of all controls, easier transition between irrigation/suction and electrocautery modes, and more control of how far a monopolar electrode tip is exposed.

Embodiments with Valve Manifold

In one embodiment, an electrosurgical device comprises a fluid flow manifold with valves to make the operation of fluid flow control buttons easier to press during a surgical procedure. In one embodiment, for example, the fluid flow control buttons control the operation of irrigation and suction valves. The valves may be stopcock valves or other types of valves that are operatively attached to the buttons. The control buttons convert linear motion to rotational motion to rotate the valves enough to open and close them. The stopcock valves require low force required to push the buttons as well as the travel needed. Also, a trigger is provided to extend and retract the shaft knob. The trigger may be attached to a lever arm so that the motion of the trigger matches the motion of the knob. In one embodiment, when the trigger is out (distal) the shaft knob is out (distal) and when the trigger is in (proximal) the shaft knob is in (proximal). The motion matching lever arm may be attached to a slide. The slide may be attached to the shaft as well as to an electrosurgical probe electrode. The trigger also may be attached to a spring to allow for easy return of the trigger to its starting position (e.g., from a proximal position when the trigger is squeezed by the operator to a distal position when the operator releases the trigger). Accordingly, the controls for operating the electrosurgical treatment, cauterization, and/or fluid flow functions, such as irrigation/suction functions, for example, may be comfortable and easy to use for the operator. In one embodiment, an energy button used to activate the electrosurgical function may be located on top of the handle assembly and the irrigation/suction buttons may be located in front of the handle assembly such that when the trigger is extended outwardly (distal) it makes is easier for the operator to press the irrigation/suction buttons. When the trigger is located in a retracted inwardly position (proximal) it makes it easier for the operator to actuate the energy button and the suction button simultaneously. In addition, the disclosed embodiments enable the control elements (e.g., irrigation/irrigation buttons, energy switch, trigger, etc.) to be manipulated with one hand.

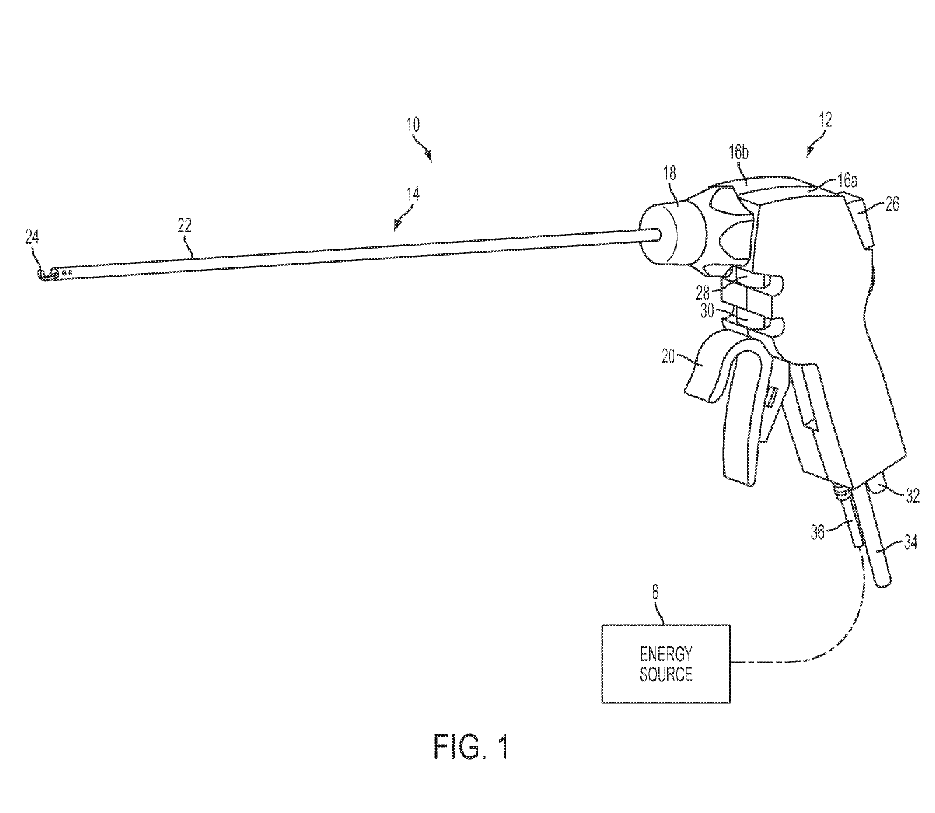

In one embodiment, an electrosurgical device comprises suction, irrigation, and energy delivery combination with an extending and retracting shaft coupled to a movement element of a trigger. The motion and/or direction of the trigger matches the motion and/or direction of the moving shaft. The trigger may couple to the shaft through a set of lever arms and/or cam surfaces. The trigger may couple to the shaft through a set of gears. In another embodiment, the trigger is spring returned. The suction and irrigation valves may be rotational stopcocks coupled to linear motion buttons. In various embodiments, the energy delivery system of the electrosurgical device may employ radio frequency (RF) energy to treat and/or cauterize tissue in monopolar or bipolar energy modes. In various embodiments, the handle assembly may have a pistol grip configuration, a pencil grip configuration, or may be configured to articulate between a pistol grip and a pencil grip and vice-versa. These embodiments are described hereinbelow in connection with FIGS. 1-31.

FIG. 1 depicts a perspective view of an electrosurgical device 10 comprising a handle assembly 12, a shaft portion 14, and an electrode 24. The electrosurgical device 10 comprising irrigation and suction features and a slidably movable element to control exposure of the electrode portion 24 of the electrosurgical device 10, according to one embodiment.

The handle assembly 12 is configured as a pistol grip and comprises left and right handle housings or shrouds 16a, 16b, a rotatable shaft knob 18, a trigger 20, an energy button 26 or switch, a first flow control button, e.g., an irrigation button 28, and a second flow control button, e.g., a suction button 30. Irrigation and suction tubes 32, 34 and an electrical cable 36 enter the handle assembly 12 through a bottom portion. The shaft portion 14 comprises a slidable sheath 22 that extends distally and retracts proximally to respectively conceal and expose the electrode portion 24. The slidable sheath 22 is operatively coupled to the trigger 20. The irrigation and suction tubes 32, 34 are fluidically coupled to a manifold and respective valves located within the space created between the left and right housing portions 16a, 16b of the handle assembly 12. The irrigation and suction buttons 28, 30 control stopcock valves located within the manifold to control the flow of irrigation fluid through the irrigation tube 32 and suction through the suction tube 34. The electrical cable 36 is electrically coupled to the energy switch 26 and an energy source 8. The energy source 8 may be a monopolar or bipolar RF energy source. The energy source 8 may be suitable for therapeutic tissue treatment as well as tissue cauterization/sealing. The energy switch 26 controls the delivery of energy to the electrode 24. A detailed explanation of each of these control elements is provided hereinbelow. As used throughout this disclosure, a button refers to a switch mechanism for controlling some aspect of a machine or a process. The buttons may be made out of a hard material such as usually plastic or metal. The surface may be formed or shaped to accommodate the human finger or hand, so as to be easily depressed or pushed. Buttons can be most often biased switches, though even many un-biased buttons (due to their physical nature) require a spring to return to their un-pushed state. Terms for the “pushing” of the button, may include press, depress, mash, and punch.

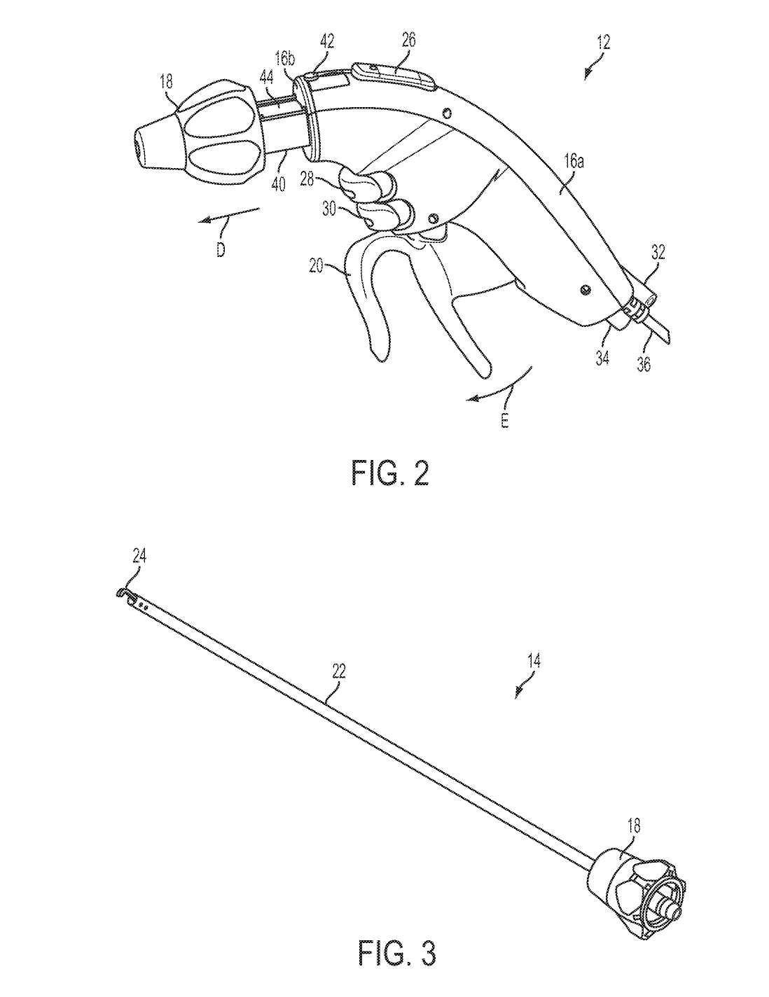

FIG. 2 depicts a perspective view of the handle assembly 12 of the electrosurgical device 10 shown in FIG. 1, according to one embodiment. The shaft knob 18 of the handle assembly 12 shown in FIG. 2 is extended distally along a slide element 40, which encases a nozzle 44 fluidically coupled to a manifold located within the handle assembly 12. As will be described in more detail below, the slide element 40 element is operatively coupled to the trigger 20. The trigger 20 is shown extended in a first “initial” position (e.g., extended distally in direction E) under the influence of a biasing element such as a spring. The position of the trigger 20 shown in FIG. 2 may be referred to herein as the initial or original position in which the trigger 20 is normally configured in and may automatically return to unless a trigger lockout mechanism is provided to prevent automatic return to the initial position. A shaft lock button 42 is provided to lock the shaft portion 14 (FIG. 1) in place. The shaft knob 18 and slide element 40 are operatively coupled to the slidable sheath 22 (FIG. 1) to conceal and/or expose the distal electrode 24 (FIG. 1).

FIG. 3 depicts a perspective view of the shaft portion 14 of the electrosurgical device 10 show in FIG. 1 configured to couple to the handle assembly 12 of the electrosurgical device 10 shown in FIG. 1, according to one embodiment.

With reference to FIGS. 1-3, the slidable sheath 22 is coupled to the shaft knob 18 at a proximal end and is slidable longitudinally to control the exposure of the electrode 24. In one embodiment, when the trigger 20 (FIG. 2) is extended outwardly in a distal position, the slidable sheath also is extended outwardly in a distal position to conceal the electrode 24. In this mode, the electrode 24 is isolated from tissue and thus the application of energy to the electrode 24 does not affect the tissue. In the same embodiment, when the trigger 20 is squeezed by the operator inwardly in a proximal position, the slidable sheath 22 is retracted to expose the electrode 24. Once the electrode 24 is exposed, the energy switch may be activated to apply energy to any tissue in contact with the electrode 24. In other embodiments, the operation may be opposite of that described above in connection with the disclosed embodiment such that when the trigger 20 is extended distally, the electrode 24 is exposed and when the trigger 20 is squeezed proximally, the electrode 24 is concealed.

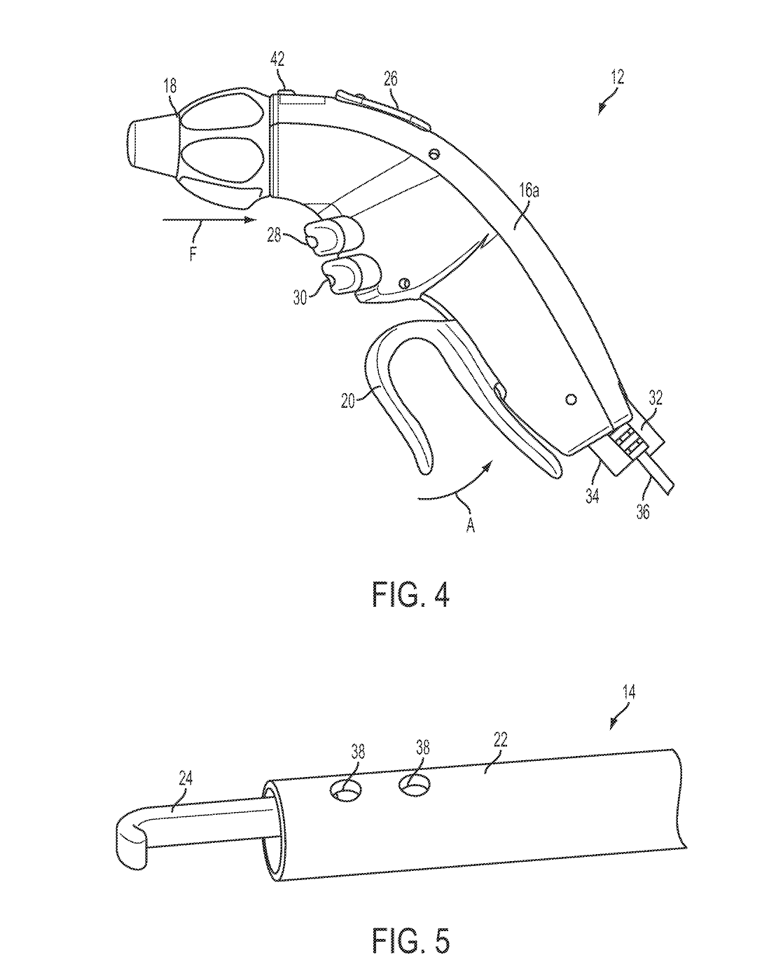

FIG. 4 depicts a side elevational view of the handle assembly 12 of the electrosurgical device 10 shown in FIG. 1 where the trigger 20 portion is shown in a second position (e.g., squeezed proximally in direction A) such that the slidably movable element, e.g., the slidable sheath 22 (FIGS. 1 and 3) is in a retracted position, according to one embodiment. When the trigger 20 is in the extended position the slidable sheath 22 is retracted proximally. This position enables the energy to be applied to the distal electrode 24 (FIGS. 1 and 3) by activation of the energy switch 26.

FIG. 5 depicts a distal end view of the shaft portion 14 of the electrosurgical device shown in FIG. 1 that is coupled to the handle assembly 12 of the electrosurgical device 10 shown in FIG. 4, where the slidably movable sheath 22 is in the retracted position to expose the electrode 24, according to one embodiment. The distal portion of the slidable sheath 22 also defines fluid flow ports 38 (e.g., irrigation/suction flow ports) delivering fluids to the surgical site or suctioning fluid therefrom.

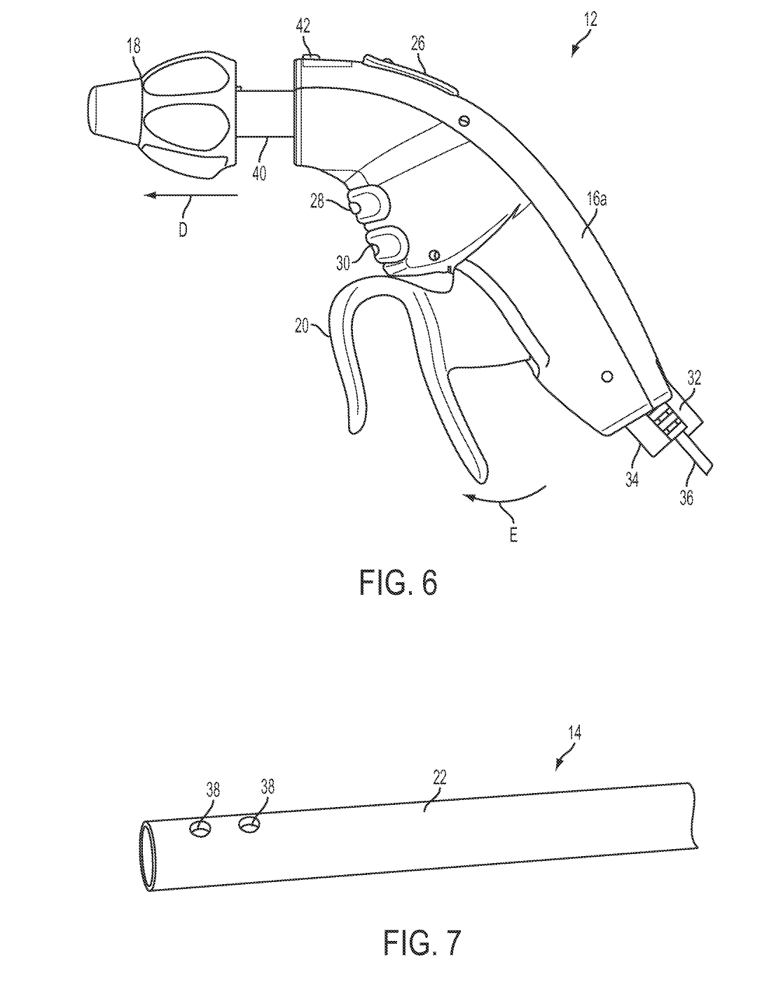

FIG. 6 depicts a side elevational view of the handle assembly 12 of the electrosurgical device 10 shown in FIG. 1 where the trigger 20 is in the first position (e.g., extended distally in direction E) such that the slidably movable element, e.g., the slidable sheath 22 (FIGS. 1 and 3) is in an extended position in direction D to conceal the electrode 24, according to one embodiment. The trigger 20 is sprung forward distally to enter the different fluid flow modes (e.g., irrigation/suction modes). The nozzle 44 (FIG. 2) moves forward distally over the electrode 24 tip to conceal it. In one example, the irrigation and suction fluid flow control buttons 32, 34 are shown pressed in with a predetermined travel distance. In one embodiment, the irrigation and suction buttons 32, 34 may travel up to 2 inches.

FIG. 7 depicts a distal end view of the shaft portion 14 of the electrosurgical device shown in FIG. 1 that is coupled to the handle assembly 12 of the electrosurgical device 10 shown in FIG. 6, with a slidably movable sheath 22 in the extended position to conceal the electrode 24, according to one embodiment. In the irrigation/suction mode, the irrigation/suction fluid flow ports 38 are employed to deliver fluids to the surgical site or suction fluid therefrom.

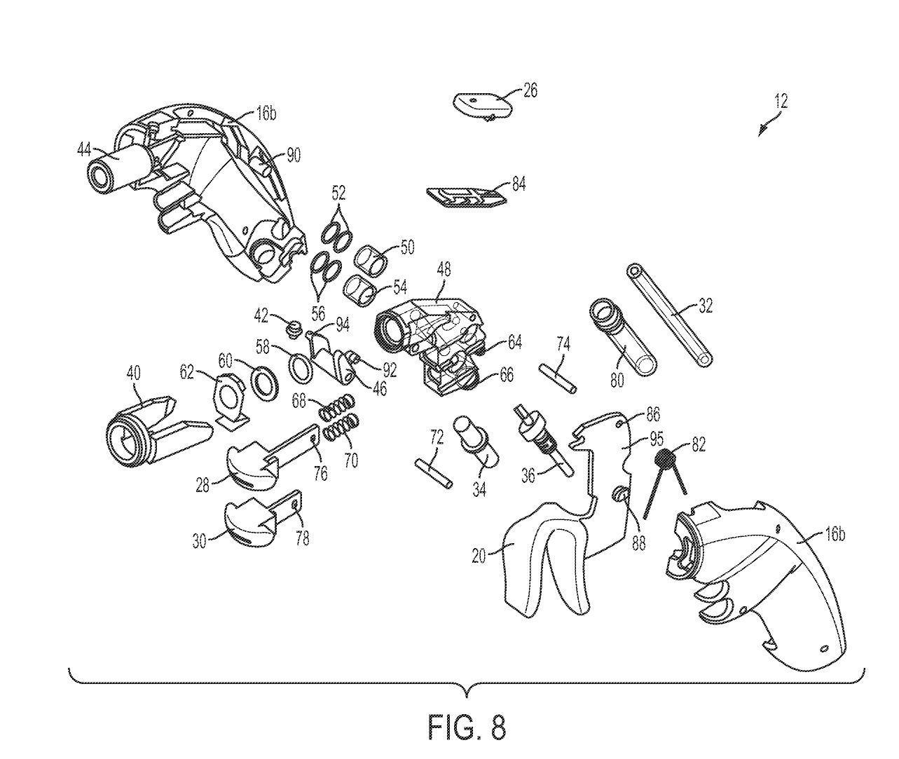

FIG. 8 depicts an exploded view of the handle assembly 12 of the electrosurgical device 10 shown in FIG. 1, according to one embodiment. This view shows the components of the valve manifold located within the handle assembly 12. The left and right shrouds 16a, 16b (handle housings) provide a support for the irrigation and suction fluid flow control buttons 28, 30, the trigger 20, and the slide element 40. In addition, the left and right shrouds 16a, 16b also support a valve manifold 48 that is fluidically coupled to the irrigation and suction ports 32, 34 as well as the nozzle 44. The left and right shrouds 16a, 16b also support the energy switch 26 and a circuit board element 84 as well as the electrical cable 36.

In one embodiment, the valve manifold 48 is configured to rotatably support an irrigation valve 50 and a suction valve 54. Although the irrigation and suction valves 50, 54 are stopcock valves, other suitable valves may be employed without departing from the scope of the present disclosure. Irrigation valve O-rings 52 are located about grooves provided on the outer surface of the irrigation valve 50. Suction valve O-rings 56 are located about grooves provided on the outer surface of the suction valve 54. The irrigation and suction valves 50, 54 include tabs or drive dogs that engage respective slots 76, 78 formed on linear arm portions of the respective irrigation and suction buttons 28, 30. The valve manifold 48 also includes an O-ring 58 and a washer 60. A retainer clip 62 that also acts as an electrical spring contact retains the valve manifold 48 to the right shroud 16b. Irrigation and suction ports 64, 66 are used to fluidically couple the valve manifold 48 to the irrigation tube 32 and suction hose 80, which is fluidically coupled to the suction tube coupling 34. The valve manifold 48 is supported within the handle assembly 12 by a pin 74 which is received within corresponding holes formed in each shroud 16a, 16b.

The irrigation and suction buttons 28, 30 (flow control buttons) each include a spring 68, 70 to bias and return the buttons 28, 30 to the distal position after being actuated.

A reversing lever arm 46 is pivotally coupled to the right shroud 16a, 16b via a pin 72. A first projecting tab 94 is received within a corresponding hole in the right shroud 16b and a second projecting tab 92 is engaged by a corresponding notch 96 formed in a lever arm 95 portion of the trigger 20. The lever arm 95 includes a pivot hole 86 about which the trigger 20 rotates. The reversing lever arm 46 is operatively coupled to the slide knob 40, which is operatively coupled to the slidable sheath 22 (FIGS. 1, 3, 5, 7) of the shaft portion 14 (FIGS. 1, 3, 5, 7). The notch 96 in the lever arm 95 of the trigger 20 engages the second projecting tab 92 of the reversing lever arm 46 to cause the motion of the slide knob 40 to match the motion of the trigger 20, e.g., trigger 20 forward—slide knob 40 forward and trigger 20 backward—slide knob 40 backward. The slide knob 40 is slidably movable over the nozzle 44, which is fluidically coupled to the output port of the valve manifold 48. A torsion spring 82 is located over a hub 90 formed in the right shroud 16b. One arm of the torsion spring 82 is coupled to a slot 88 formed in the lever arm 95 portion of the trigger 20 and another arm of the torsion spring 82 is located against a back wall of the right shroud 16b to bias the trigger 20 outwardly in a distal position when not squeezed and to return the trigger 20 outwardly to the distal position when the operator releases the trigger 20.

The circuit board 84 is mounted to the right shroud 16b. The energy switch 26 is electrically coupled to the circuit board 84. The electrical spring contact 62 also is electrically coupled to the circuit board 84. The electrical spring contact 62 is electrically coupled to the electrode 24 (FIGS. 1, 3, 5). The electrical cable 36 couples energy from the energy source to the circuit board 84. When the energy switch 26 is activated energy is coupled from the energy source to the electrode 24 via the electrical spring contact 62.

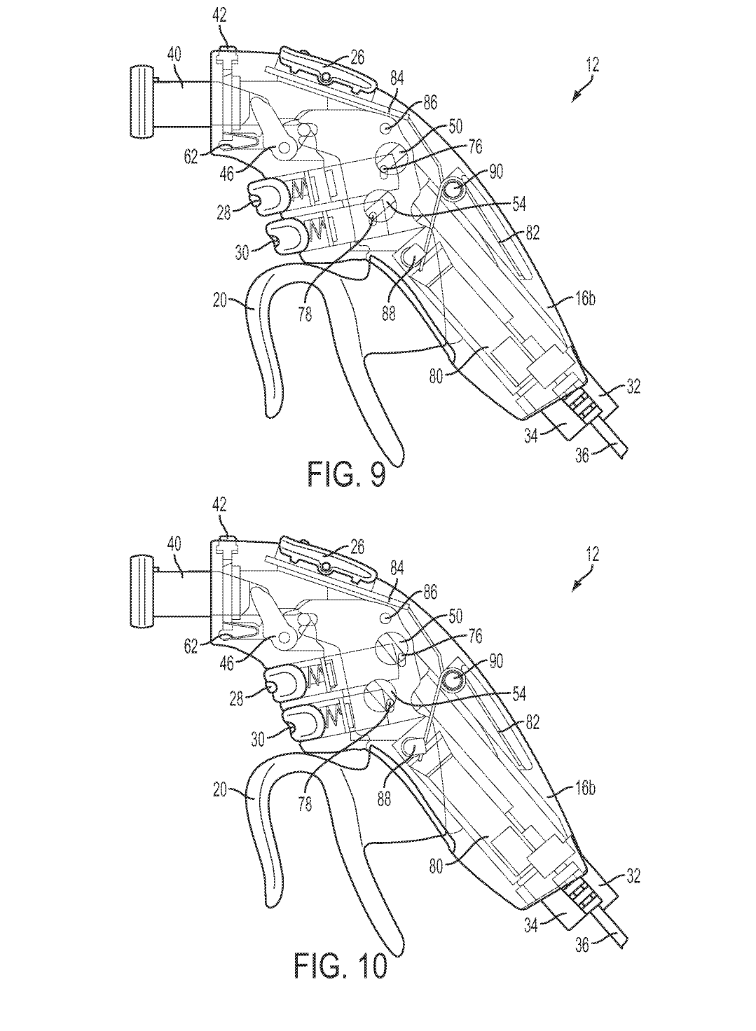

FIG. 9 depicts a partial cut-away transparent side elevational view of the handle assembly 12 of the electrosurgical device 10 shown in FIG. 1 with the irrigation and suction buttons 28, 30 pushed out by the corresponding irrigation and suction button springs 68, 70 to disable fluid flow, according to one embodiment.

FIG. 10 depicts a partial cut-away transparent side elevational view of the handle assembly 12 of the electrosurgical device 10 shown in FIG. 1 with the irrigation and suction buttons 28, 30 pushed in to enable fluid flow, according to one embodiment.

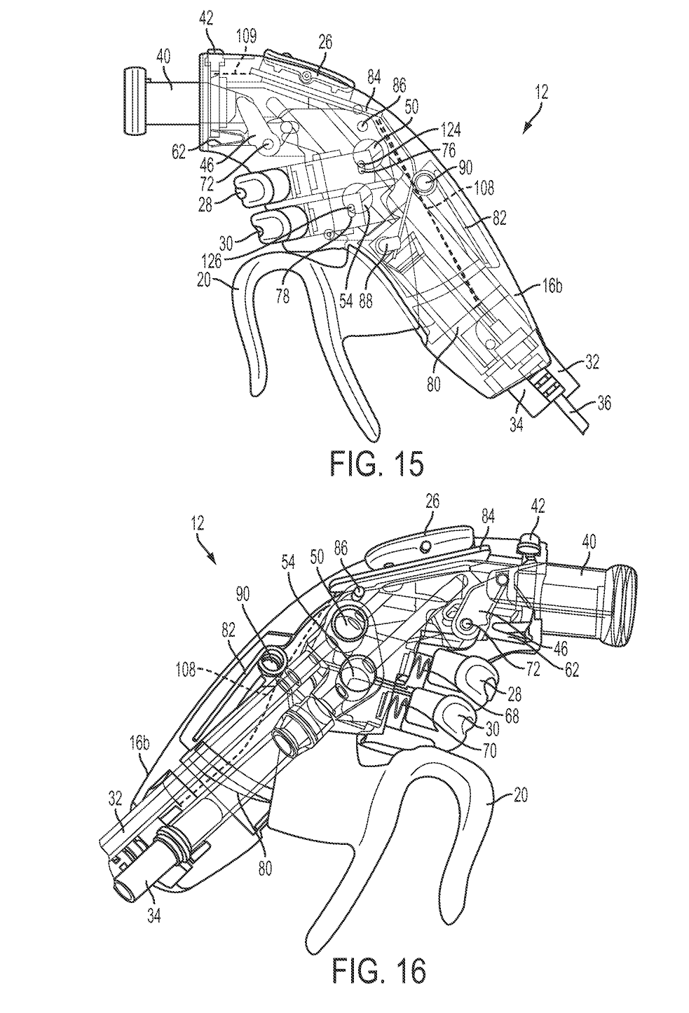

With reference now to FIGS. 9 and 10, the slots 76, 78 formed in the arm portions of the irrigation and suction buttons 28, 30 engage projecting tabs 124, 126 (FIGS. 15, 17, 18) on the stopcock valves 50, 54. When the buttons 28, 30 are positioned outwardly as shown in FIG. 9, the valves 50, 54 are positioned in a closed position to block flow. When the buttons 28, 30 are pressed, the slots 76, 78 act on the corresponding tabs 124, 126 on the valves 50, 54 and rotate the valves 50, 54 from a closed position to an open position as shown in FIG. 10 to enable flow through the valves 50, 54.

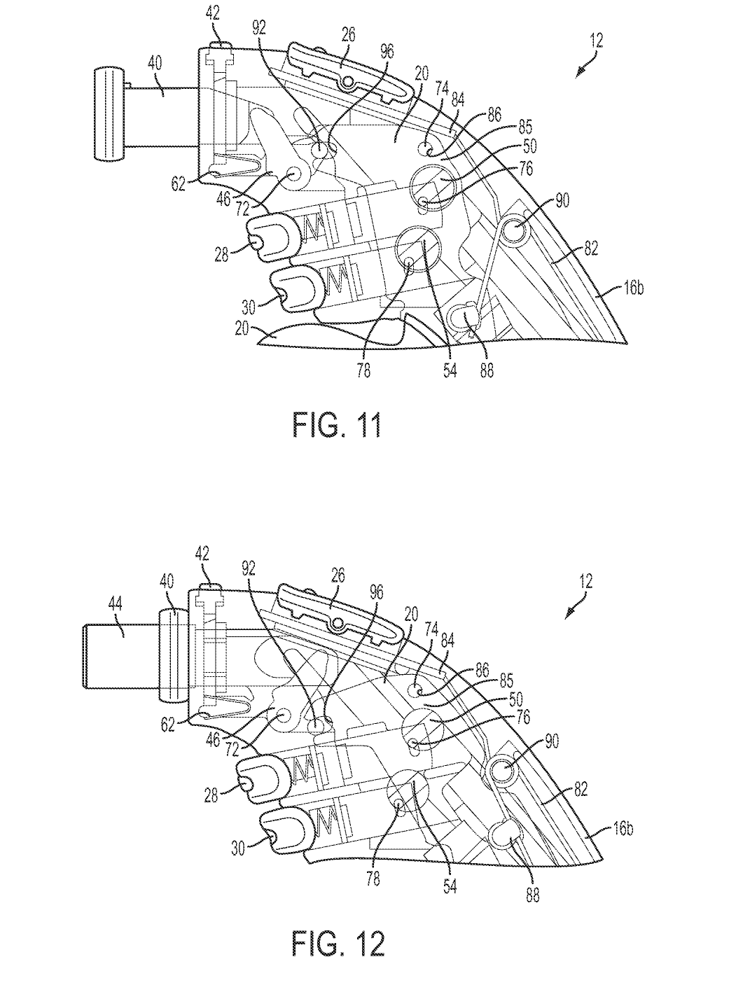

FIG. 11 depicts a partial cut-away transparent side elevational view of the handle assembly 12 of the electrosurgical device 10 shown in FIG. 1 with the reversing lever arm 46 operatively coupled to the trigger 20 where the trigger 20 is located in a forward (distal) position as biased by the torsion spring 82 to extend the slide element 40 forward in a distal direction to conceal the electrode 24 (FIGS. 1, 3, 5), according to one embodiment. The torsion spring 82 returns the trigger 20. The torsion spring 82 is located over the hub 90 and one arm is coupled to the lever arm 85 portion of the trigger 20 at the slot 88 and another arm is positioned against a hard wall portion of the right shroud 16b to provide resistance when the trigger 20 is squeezed. As shown in FIG. 9, the torsion spring 82 is in tension mode to bias the trigger outwardly in a distal direction. The reversing lever arm 46 is pivotally coupled to the right shroud 16b by the pin 72. The lever arm 85 portion of the trigger 20 is pivotally coupled at pivot 86 to the right shroud 16b by the pin 74. The notch 96 formed in the lever arm 85 portion of the trigger 20 engages the projecting tab 92 of the reversing lever arm 46. As shown in FIG. 9, the reversing lever arm 46 applies a pushing force to the slide knob 44 to maintain the slidable sheath 22 (FIG. 7) in a distal direction to conceal the electrode 24.

FIG. 12 depicts a partial cut-away transparent side elevational view of the handle assembly 12 of the electrosurgical device 10 shown in FIG. 11 with the trigger 20 in a backward (proximal) position squeezed to overcome the bias force of the torsion spring 82 to retract the slide element 40 backward in a proximal direction to expose the electrode 24 (FIGS. 1, 3, 5), according to one embodiment. As the trigger 20 is squeezed, the lever arm 85 portion rotates counterclockwise about the pivot point 86 and the notch 96 applies a pushing force to the projecting tab 92 of the reversing lever arm 46 causing the reversing lever arm 46 to rotate clockwise applying a pulling force to the slide knob 44 to retract the slidable sheath 22 (FIG. 1, 3, 5) in a proximal direction to expose the electrode 24 and enable energy to be applied to tissue contacting the electrode 24 at the surgical site.

With reference to FIGS. 11 and 12, the reversing lever arm 46 enables the motion direction of the trigger 20 to match the motion direction of the slide knob 44, and hence, the motion direction of the slidable sheath 22. In accordance with the illustrated embodiment, when the trigger 20 is forward the slide knob 40 is forward and when trigger 20 is back the slide knob 44 is back.

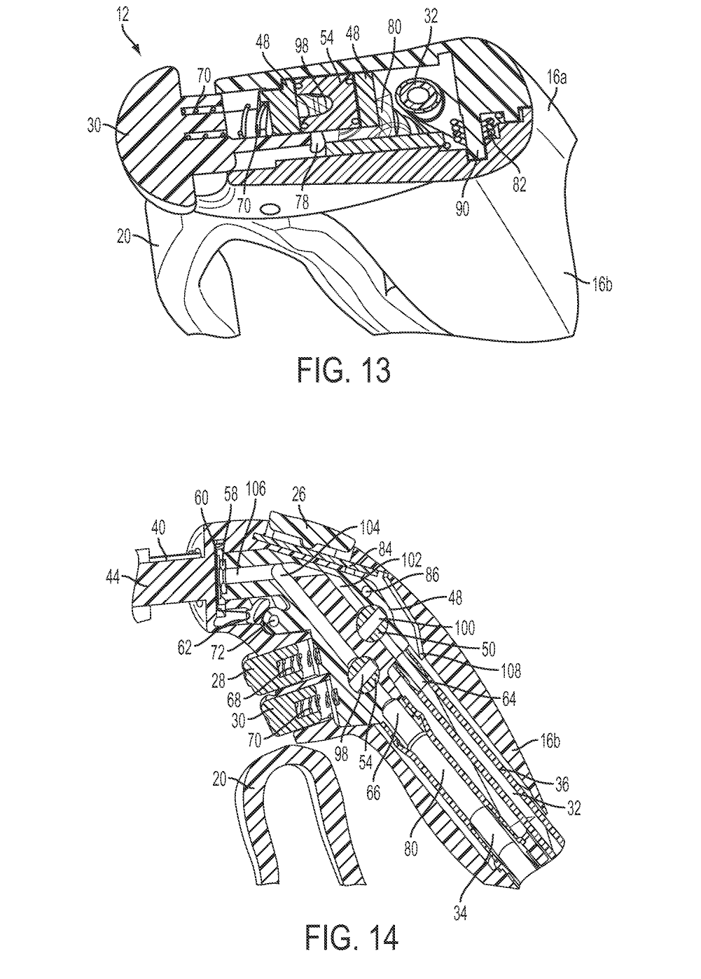

FIG. 13 depicts a transverse sectional view of the handle assembly 12 of the electrosurgical device 10 shown in FIG. 1 to further illustrate the operation of the irrigation and suction valves 50, 54, according to one embodiment. As shown in FIG. 13, when the suction valve 54 is pushed to the far right by squeezing the suction button 30, the force required to rotate the valve 54 within the valve manifold 48 and open the suction valve port 98 is centered on the button 30. The irrigation button/irrigation valve operates in a similar manner.

FIG. 14 depicts a longitudinal sectional view of the handle assembly 12 of the electrosurgical device 10 shown in FIG. 1 to further illustrate the operation of the irrigation and suction valves 50, 54, according to one embodiment. The valve manifold 48 is fluidically coupled to the irrigation tube 32 via the irrigation port 64 and to the suction tube 80 via the suction port 66. The valve manifold 48 includes an irrigation channel 102 and a suction channel 104. The irrigation tube 32 is fluidically coupled to the irrigation channel 102 via the irrigation valve 50. When the irrigation button 28 is depressed, the irrigation valve 50 rotates such that the irrigation valve port 100 fluidically couples the irrigation tube 32 to the irrigation channel 102 and causes irrigation fluid to flow to the main flow channel 106 and through the nozzle 44 down to the irrigation ports 38 at the distal end of the shaft portion 14 (FIGS. 3, 5). When the irrigation button 28 is released (as shown), the irrigation valve 50 blocks the flow. The suction tube 80, 34 is fluidically coupled to the suction channel 104 via the suction valve 54. When the suction button 30 is depressed, the suction valve 54 rotates such that the suction valve port 108 fluidically couples the suction tube 80, 34 to the suction channel 104 and causes suction fluid to flow from the irrigation ports 38 at the distal end of the shaft portion 14 through the nozzle 44 and the main flow channel 106 through the suction channel 104, the suction valve port 109, and the suction tubes 80, 34.

FIG. 15 depicts a partial cut-away transparent view of the handle assembly 12 of the electrosurgical device 10 shown in FIG. 1 to illustrate electrical wiring connections between an energy cable 36, an energy switch circuit board 84 of the energy switch 26, and an electrical contact spring 62 according to one embodiment.

FIG. 16 depicts a right side perspective view of the handle assembly 12 of the electrosurgical device 10 shown in FIG. 15 to illustrate the wiring between the energy cable 36 and the circuit board 84 of the energy switch 26, according to one embodiment.

With reference to both FIGS. 15 and 16, the energy cable 36 coupled to the energy source enters through a bottom portion of the handle assembly 12. Electrical wires 108 from the cable 36 are routed to and electrically connected to the circuit board 84. The energy switch 26 is electrically connected to the circuit board 84. Another electrical wire 109 is electrically connected between the circuit board 84 and the electrical contact spring 62. The electrical contact spring 62 is electrically connected to the electrode 24 (FIGS. 1, 3, 5).

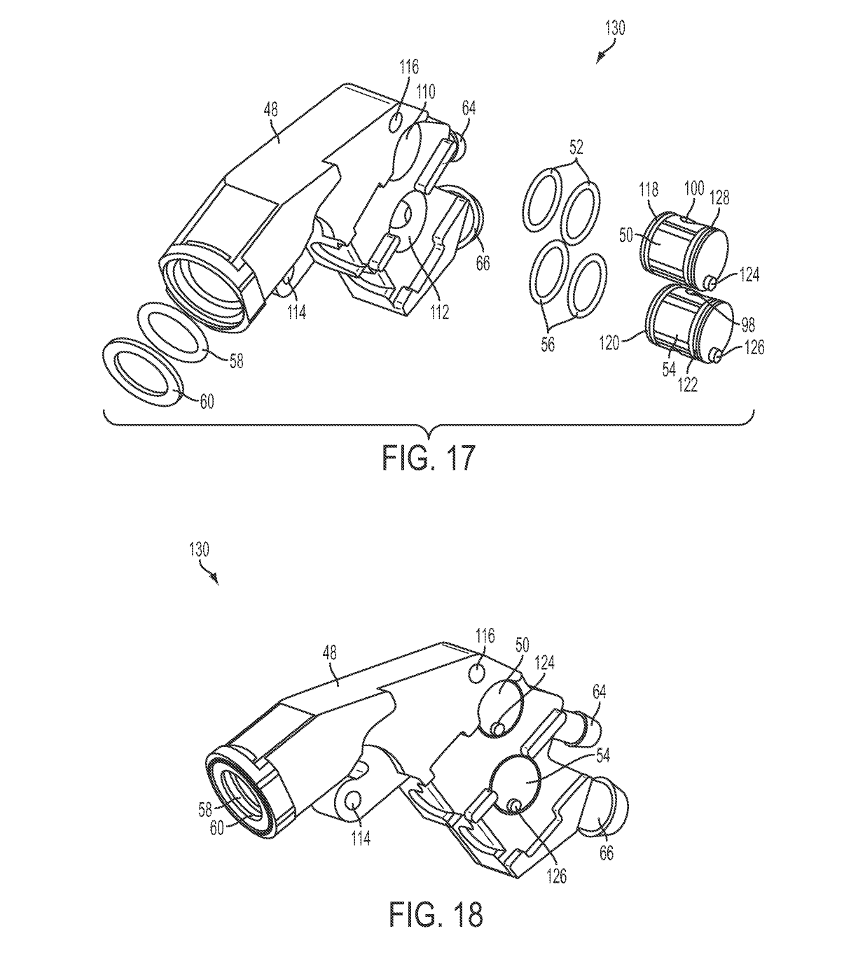

FIG. 17 depicts an exploded view of a valve manifold assembly 130, according to one embodiment. The valve manifold assembly 130 comprises a valve manifold 48, valves 50, 54, and seals. The valve manifold 48 includes irrigation and suction ports 64, 66 to fluidically couple the valve manifold assembly 130 to irrigation and suction tubes. The valve manifold 48 also defines apertures 110, 112 to rotatably receive the respective irrigation and suction valves 50, 54 therein. O-ring seals 52 are located over grooves 118, 128 formed about an outer surface of the irrigation valve 50. O-ring seals 56 are located over grooves 120, 122 formed about an outer surface of the suction valve 54. The irrigation valve 50 includes an irrigation valve port 100 and the suction valve 54 includes a suction valve port 98. Each valve 50, 54 also includes a projecting tab 124, 126 that engages corresponding slots 76, 78 in the arm portions of the irrigation and valve buttons 28, 30 (FIGS. 8-12, 15).

Once the O-rings 52, 56 are applied on the valves 50, 54 the valves 50, 54 are inserted into the valve apertures 110, 112 of the valve manifold 48. The O-rings 52, 56 may be omitted when using solid valves. O-ring 58 is inserted into a port of the valve manifold and a washer 60 is welded into place over the O-ring 58.

FIG. 18 depicts a perspective view of an assembled valve manifold assembly 130 shown in FIG. 17, according to one embodiment. Mounting holes 114, 116 are provided to mount the valve manifold assembly 130 within the handle assembly 12.

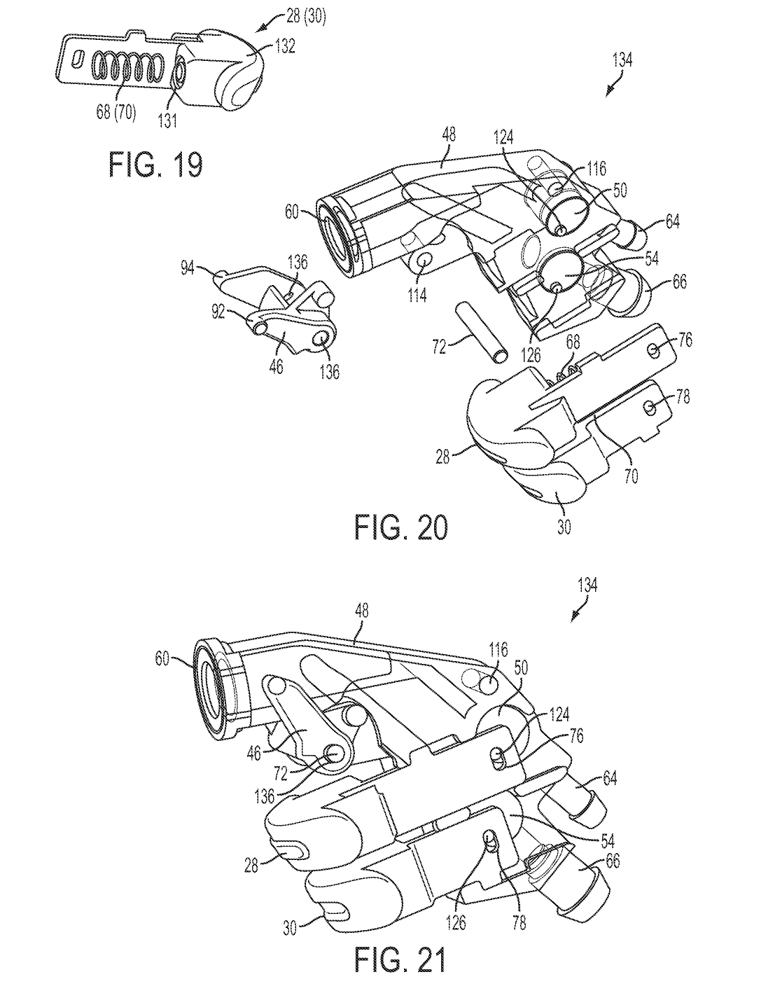

FIG. 19 depicts a button 28, 30 to operate either the irrigation and/or suction functions, according to one embodiment. The button 28, 30 comprises a button body 132 and a corresponding spring 68, 70. The spring 68, 70 is inserted into an aperture 131 defined in the button body 132. The spring 68, 70 returns the button body 132 back to its initial position.

FIG. 20 depicts an exploded view of a “valve manifold assembly/buttons/reversing arm” assembly 134, according to one embodiment. The reversing lever arm 46 is pivotally coupled to the valve manifold 48 by a pin 72 inserted through a mounting hole 136 defined in the reversing lever arm 46 and through the mounting hole 114 defined in the body of the valve manifold 48. The pin 72 locks the reversing lever arm 46 onto the valve manifold 46. The irrigation and suction buttons 28, 30 are inserted onto the valve manifold 46. The springs 68, 70 are aligned with the valve slots 76, 78 to corresponding features. The irrigation and suction buttons 28, 30 are operatively coupled to the corresponding irrigation and suction valves 50, 54 by inserting the valve slots 76, 78 to the corresponding protruding tabs 124, 126 of the valves 50, 54.

FIG. 21 depicts a perspective view of the assembled “valve manifold assembly/buttons/reversing arm” assembly 134 shown in FIG. 20, according to one embodiment.

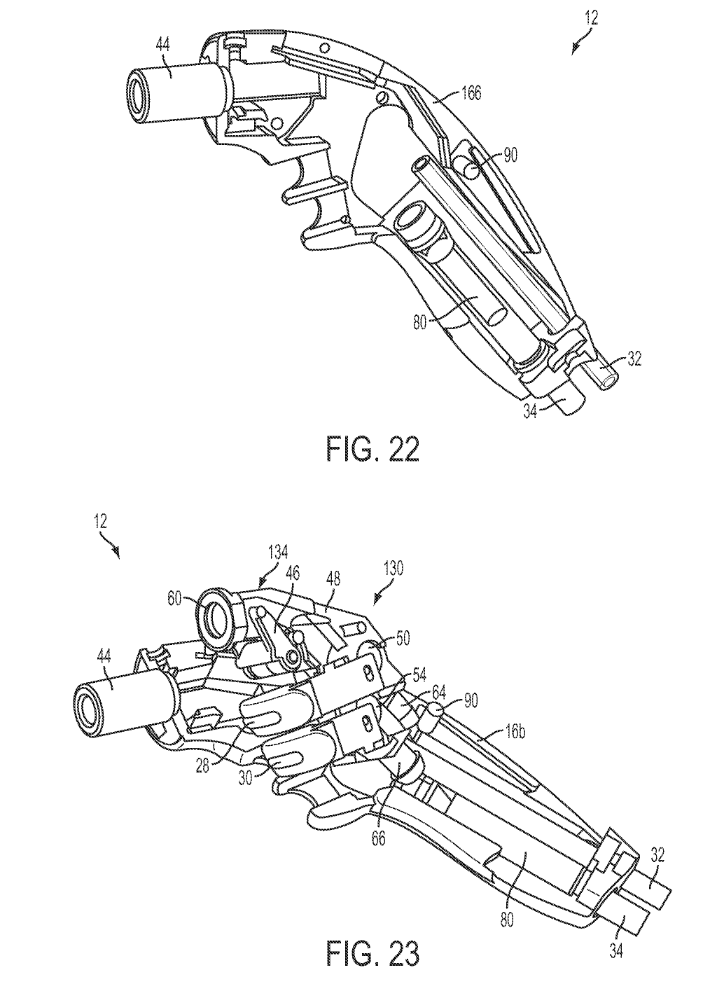

FIG. 22 depicts a perspective view of the right shroud 16b portion of the handle assembly 12 of the electrosurgical device 10 shown in FIG. 1 with the irrigation and suction hoses 32, 34 threaded through a bottom portion thereof, according to one embodiment.

FIG. 23 depicts a perspective view of the right shroud 16b portion of the handle assembly 12 shown in FIG. 22 with the “valve manifold assembly 130/buttons 28, 30/reversing lever arm 46” assembly 134 shown in FIG. 20-22 in the process of being attached to the irrigation and suction hoses 32, 34, according to one embodiment.

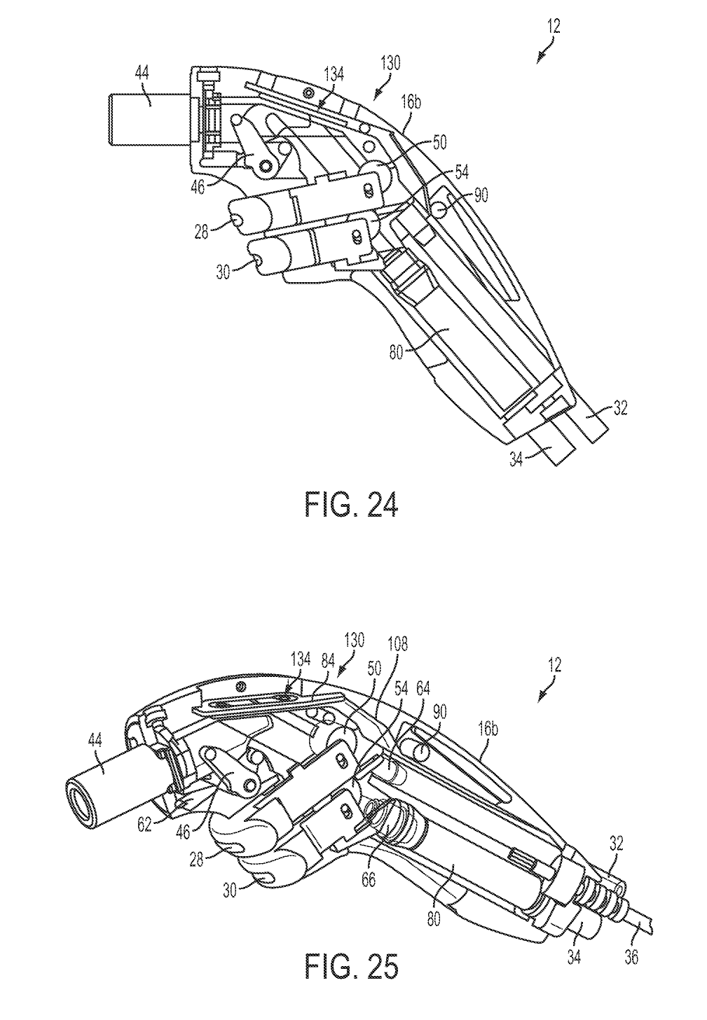

FIG. 24 depicts a side elevational view of the right shroud 16b portion of the handle assembly 12 shown in FIG. 24 with the “valve manifold assembly 130/buttons 28, 30/reversing lever arm 46” assembly 134 shown in FIG. 23 attached to the irrigation and suction hoses 32, 34, according to one embodiment.

FIG. 25 depicts a perspective view of the right shroud 16b portion of the handle assembly 12 shown in FIG. 24 with the “valve manifold assembly 134/buttons 28, 30/reversing lever arm 46” assembly 134 shown in FIG. 24 attached to the irrigation and suction hoses 32, 34 and an energy switch circuit board 84, cable 36, and retainer/electrical spring contact 62 mounted to the right shroud 16b, according to one embodiment.

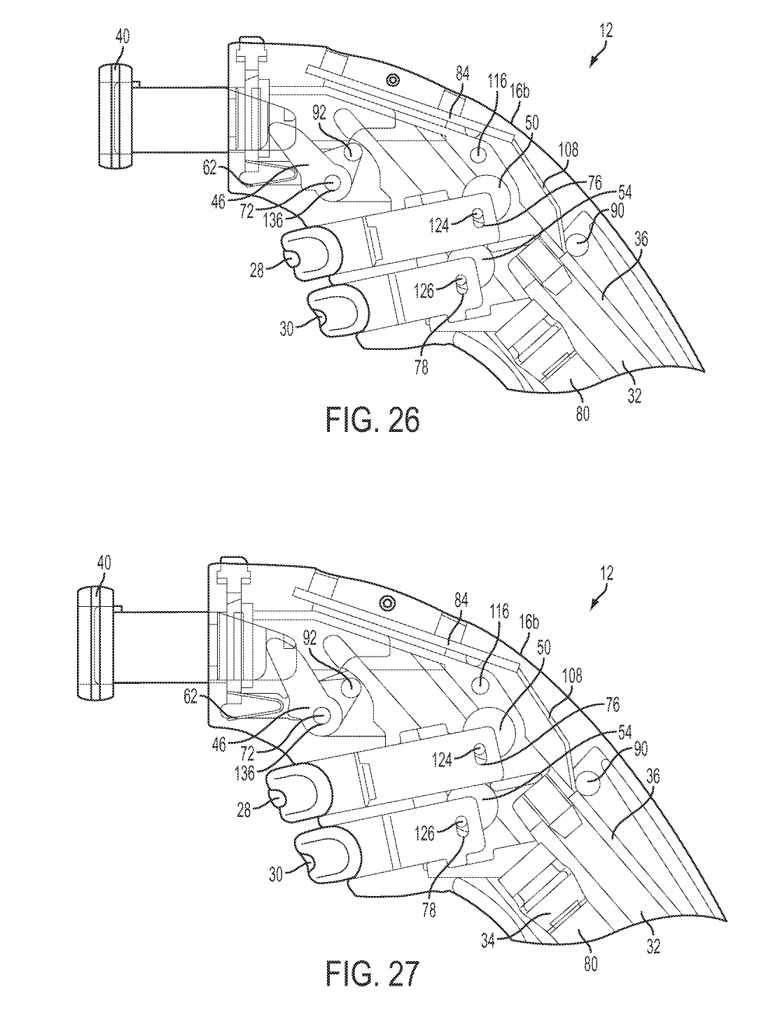

FIG. 26 depicts a partial side elevational view of the right shroud 16b portion of the handle assembly 12 shown in FIG. 25 with the reversing lever arm 46 rotated forward in a maximum distal direction and the slide element 40 slidably attached over the nozzle, according to one embodiment. As previously discussed, the slide knob 40 is operatively coupled to the reversing lever arm 46, which is operatively coupled to the trigger.

FIG. 27 depicts a partial side elevational view of the right shroud 16b portion of the handle assembly 12 shown in FIG. 26 with the reversing lever arm 46 rotated backward in a maximum proximal direction to lock the slide element 40 in place, according to one embodiment.

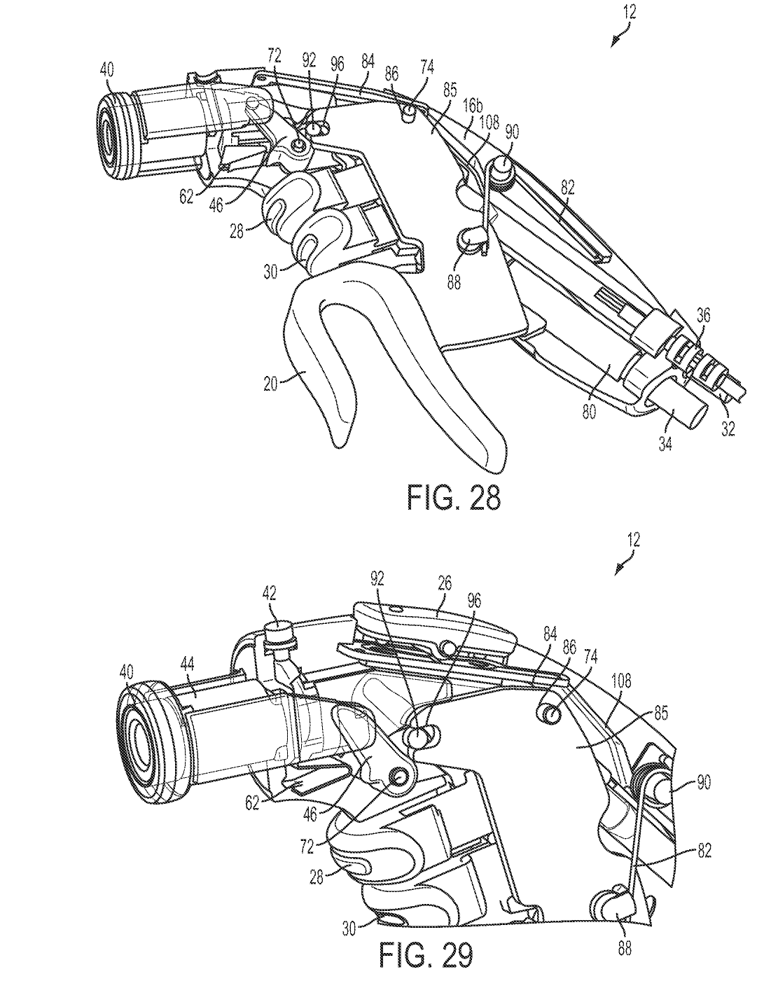

FIG. 28 depicts a perspective view of the right shroud 16b portion of the handle assembly 12 shown in FIG. 27 with the trigger 20 attached thereto and operatively coupled to the reversing lever arm 46 and the compression spring 82, according to one embodiment. The trigger 20 is inserted in the right shroud 16b and the pin 74 is inserted through the mounting hole 116. The trigger 20 lever arm body 85 is pivotally coupled to the right shroud 16b by pin 74 at pivot point 86. The compression spring 82 is compressed and inserted into the right shroud 16b and the tab 88 on the lever arm 85.

FIG. 29 depicts a partial perspective view of the right shroud 16b portion of the handle assembly 12 shown in FIG. 28 with the energy switch 26 and shaft unlock button 42 attached thereto, according to one embodiment.

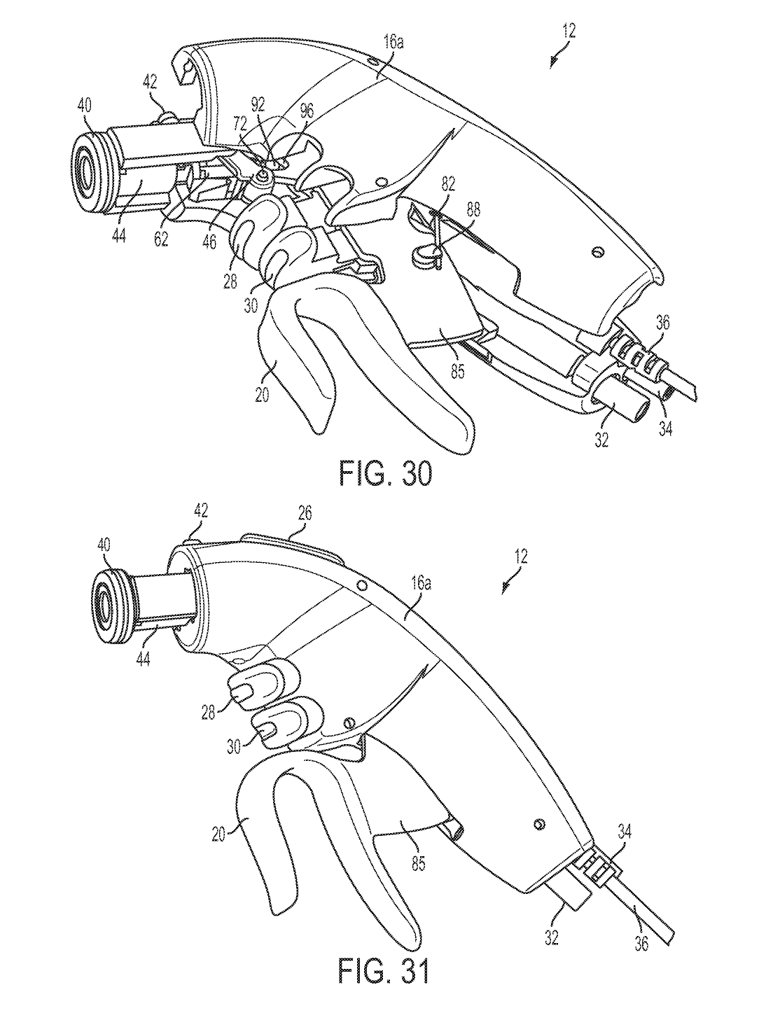

FIG. 30 depicts a perspective view of a left shroud portion 16a of the handle assembly 12 shown in FIG. 29 in the process of being attached to the right shroud 16b portion of the handle assembly 12, according to one embodiment.

FIG. 31 depicts a perspective view of an assembled handle assembly 12 of the electrosurgical device shown in FIG. 1, according to one embodiment.

Embodiments with Tripper Coupled to Cam Arm, Gear Train, or Cables

In one embodiment, the electrosurgical device may include a trigger operatively coupled to cam arm(s), gear train, or cables to move the nozzle back and forth. A sliding base comprising a slide element would attach to the nozzle. A detent lock may be used to attach the slide element to the nozzle and the handle. The slide element may include features such as a cam surface or rack to couple to the trigger. Squeezing and releasing the trigger causes the slide element to move backward and forward thus pulling and pushing the nozzle backward and forward. In certain embodiments, the trigger may include multiple detent positions for fine stopping points. It may include a lock feature to keep the trigger in a closed position such that the nozzle stays in place, and it may have a spring to automatically return the trigger and nozzle to its starting position. Thus the trigger is easy to use in a pistol grip like handle configuration all the controls can be reached with one hand and enables quick and precise low force movement of the nozzle.

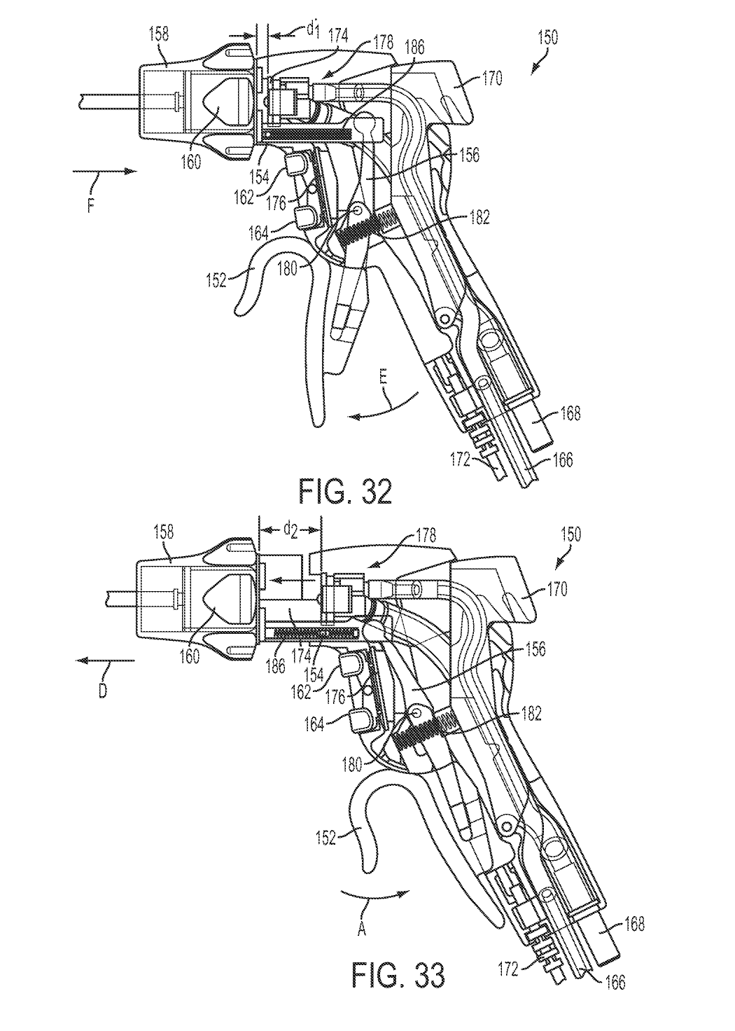

In one embodiment, an electrosurgical device comprises a sheath over a shaft that can extend over a monopolar electrode tip. The device may include a combination of RF monopolar suction and irrigation functions. The shaft may be a separate assembly that can be attached to a handle. The handle may contain a trigger coupled to a sliding feature to push the shaft forward and backward. The trigger may include a cam arm coupling to the sliding feature. The trigger may include a gear train to couple to the sliding feature. The trigger may include a cable system to couple to the sliding feature. The trigger also may include a locking feature to keep it closed in its second position. The locking feature can be a lever and spring on the trigger that automatically latches when closed. To unlock the trigger, the lever is pushed in the opposite direction from the latch. The lock also may be a close to lock then close again to unlock locking feature, which includes a spring biased member and a locking cam path. The trigger or handle may include spring biased feature(s) and detents to enable the trigger to lock in different positions. The trigger or the slide feature may include a spring coupled to them to return the trigger to its starting position automatically. In various embodiments, the electro-surgical device may employ RF energy to cauterize tissue in monopolar or bipolar energy modes. In various embodiments, the handle assembly may have a pistol grip configuration, a pencil grip configuration, or may be configured to articulate between a pistol grip and a pencil grip and vice-versa. These embodiments are described hereinbelow in connection with FIGS. 32-38.

FIG. 32 depicts a transparent side elevational view of a handle assembly 150 of an electrosurgical device comprising a trigger 152 operatively coupled to a slide element 154 by way of a cam arm 156 where the trigger 152 is extended in the distal direction E and the slide element 154 is retracted in the proximal direction F, according to one embodiment. In various embodiments, the electro-surgical device may employ RF energy to cauterize tissue in monopolar or bipolar energy modes. In various embodiments, the handle assembly 150 may include a pistol grip configuration (as shown in FIGS. 32-34), a pencil grip configuration, or may be configured to articulate between a pistol grip and a pencil grip and vice-versa.

The trigger 152 is pivotally movable about pivot 180. A return spring 182 returns the trigger 152 to its original position. In the configuration shown in FIG. 32, the trigger 152 is positioned distally and the cam arm 156 is positioned proximally. Thus, the trigger 152 attached to the cam arm 156 can move a nozzle 174 back and forth. The slide element 154 base attaches to the nozzle 174. The slide element 154 base enables the nozzle 174 to be attached to the handle assembly 150 using a detent lock 184 (shown in FIG. 34). The slide element 154 base includes features such as a cam surface or rack to couple to the trigger 152. Pulling the trigger 152 would slide the slide element 154 base forward and backward thus pushing and pulling the nozzle 174 forward and backward. In certain embodiments, the trigger 152 may include multiple detent positions for fine stopping points. The trigger 152 may include a lock feature to keep it closed so that the nozzle 174 stays in place the spring 182 automatically returns the trigger 152 and nozzle 174 to their starting position. Thus the trigger 152 is easy to use in a pistol grip like handle assembly 150 configuration all the controls can be reached with one hand and enables quick and precise low force movement of the nozzle 174. The trigger 152 or the slide element 154 may be coupled to a spring 182, 186 to return the trigger 152 and/or the slide element 154 back to its starting position automatically.

In one embodiment, an electrosurgical device comprises a shaft 160 that can extend over a monopolar electrode tip. The electrosurgical device may include a combination of RF monopolar suction and irrigation functions. The shaft 160 may be a separate assembly that can be attached to the handle assembly 150.

The handle assembly 150 comprises a rotation knob 158 operatively coupled to the shaft 160 and the slide element 154. Irrigation and suction buttons 162, 164 are operatively coupled to corresponding irrigation and suction tubes 166, 168 to control the flow of fluid and/or suction. The irrigation tube 166 and the suction tube 168 are fluidically coupled to a main flow channel 174 to deliver fluid to the surgical site or aspirate fluid and/or surgical matter from the surgical site by way of suction. The buttons 162, 164 are electrically coupled to a circuit board 176, which is coupled to a manifold assembly 178 comprising electrically controlled valves tat control the irrigation/suction functions of the electrosurgical device. An electrical button 170 or switch is electrically coupled to an electrical cable 172 and to an electrode tip located at the distal end of the shaft 160 such that activation of the electrical button 170 applies energy to an exposed electrode at the surgical site. As previously discussed in connection with the embodiments described in connection with FIGS. 1-31, the trigger 152 controls a slidable sheath that advances distally to conceal the electrode and retracts proximally to expose the electrode.

FIG. 33 depicts a transparent side elevational view of the handle assembly 150 of an electrosurgical device shown in FIG. 32 with the trigger 152 operatively coupled to the slide element 154 by way of the cam arm 156 where the trigger 152 is retracted in the proximal direction A and the slide element 154 is extended in the distal direction D, according to one embodiment. Comparing FIGS. 32 and 33, it can be seen that the slide element 154 advances a relative distance of “d2-d1” over the range of motion of the trigger 152 about the pivot 180.

FIG. 34 depicts a perspective transparent view of the handle assembly 150 of the electrosurgical device shown in FIG. 33 with the trigger 152 operatively coupled to the slide element 154 by way of the cam arm 156 where the trigger is retracted in the proximal direction A and the slide element 154 is extended in the distal direction D with the rotation knob 158 removed to show the shaft detent locking element 184, according to one embodiment. The detent locking element 184 can be employed to keep the trigger 152 closed in its second position, squeezed or triggered position. The trigger 152 or handle assembly 150 also may include spring biased feature(s) and detents to enable the trigger to lock in different positions. The trigger 152 locking element can be a lever and spring on the trigger 152 that automatically latches when closed. To unlock the trigger 152, the lever is pushed in the opposite direction from the latch. The trigger locking element also may be a close to lock then close again to unlock locking feature, which includes a spring biased member and a locking cam path.

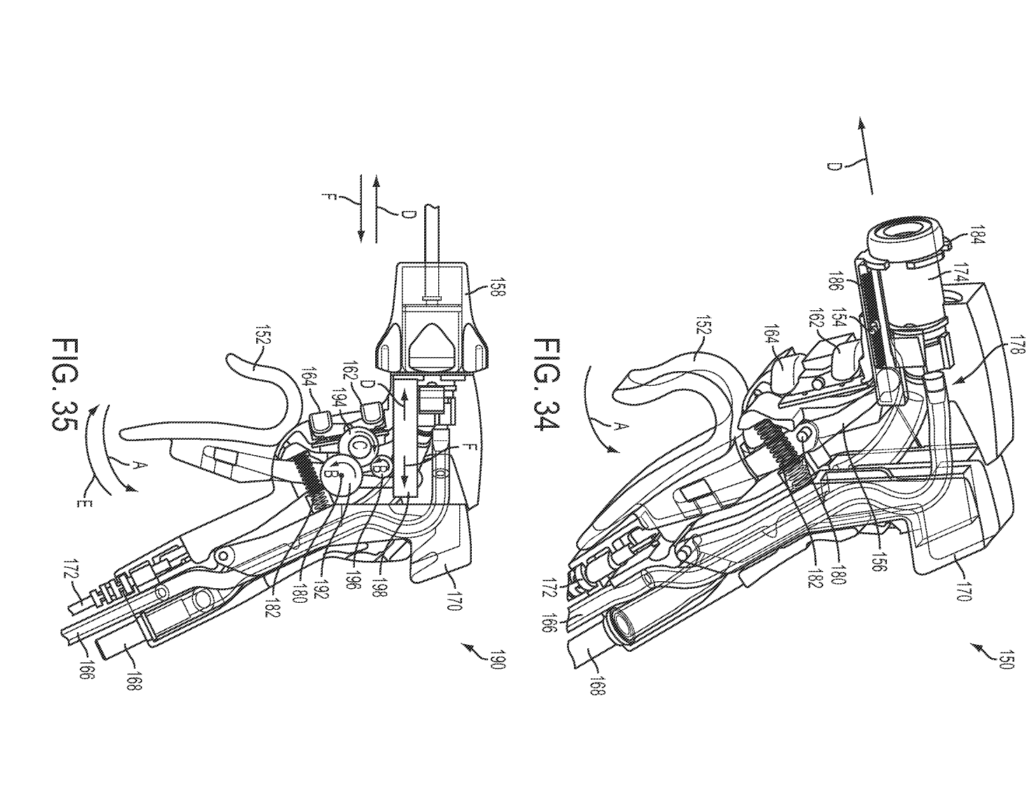

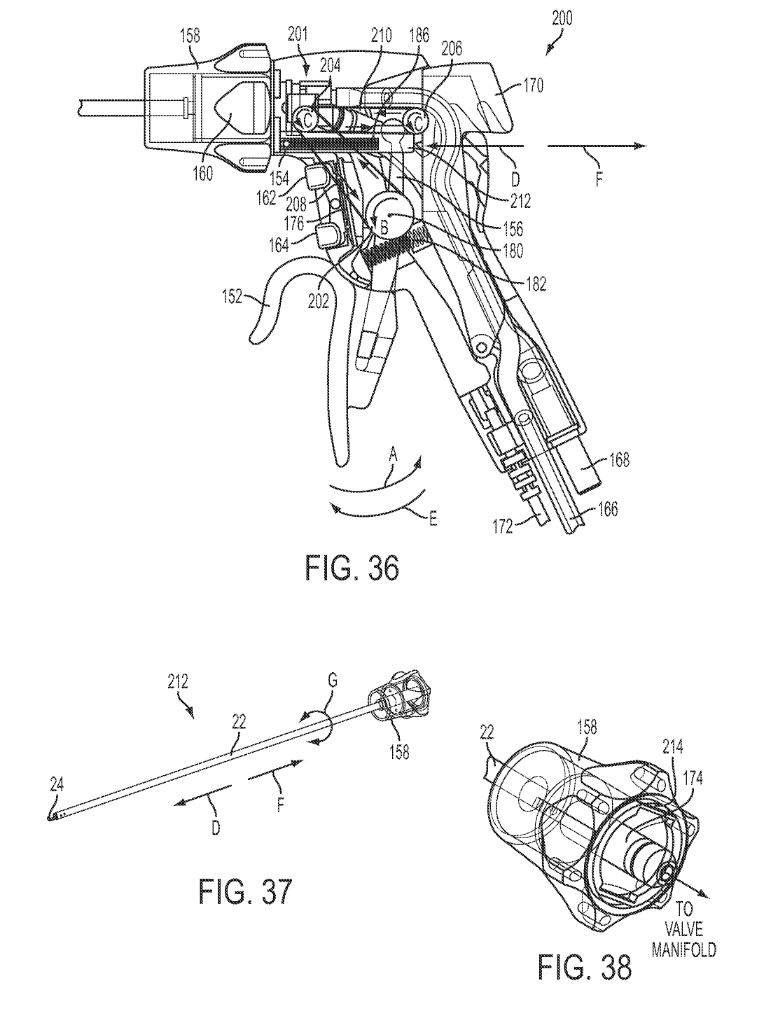

In various embodiments, the trigger 152 and slide element 154 may be operatively coupled to a gear train, cable pull system, or other force/torque amplification element or system, to reduce the force required to advance and retract the slide element 154 using the trigger 152. The embodiment described below in connection with FIG. 35 provides a handle assembly comprising a gear train operatively coupled to the trigger 152 and the slide element 154 and the embodiment described below in connection with FIG. 36 provides a handle assembly comprising a cable pull system operatively coupled to the trigger 152 and the slide element 154.

FIG. 35 depicts a transparent side elevational view of a handle assembly 190 of an electrosurgical device comprising a trigger 152 operatively coupled to a slide element 154 by way of a gear train 191 attached to a rack 198 where the trigger 152 is extended in the distal direction and the slide element 154 is retracted in the proximal direction, according to one embodiment. As shown in FIG. 35, the gear train 191 is operatively coupled to the trigger 152 and the slide element 154 to push and pull the slide element 154 forward D (distal) and backward F (proximal). The gear train 191 comprises a first gear 192 that is rotatably movable about the pivot 180. A second gear 194 is meshed with the first gear 192 and the second gear 194 is meshed with a third gear 196 that is meshed with a rack 198. The rack 198 is coupled to the slide element 154. As the trigger 152 is squeezed in direction A, the first gear 191 rotates about the pivot 180 in direction B and the second and third gears 194, 196 rotate in direction C causing the rack 198 to move distally in direction D. As the trigger 152 is returned to its original position in direction E the gear train 191 reverses direction and moves the rack 198 proximally in direction F.

FIG. 36 depicts a transparent side elevational view of a handle assembly 200 of an electrosurgical device comprising a trigger 152 operatively coupled to a slide element 154 by way of a cable pull system 201 where the trigger 152 is extended in the distal direction and the slide element 154 is retracted in the proximal direction, according to one embodiment. As shown in FIG. 36, the cable pull system 201 is operatively coupled to the trigger 152 and the slide element 154 to push and pull the slide element 154 forward D and backward F. The cable pull system 201 comprises a first pulley 202 that is rotatably movable about the pivot 180. A second pulley 204 is coupled to the first pulley 202 by a first cable 208. A third pulley 206 is coupled to the second pulley 204 by a second cable 210. The third pulley 206 is coupled to a rack 212, which is coupled to the slide element 154. In various embodiments, the third pulley 206 may comprise a gear that with the rack 212. As the trigger 152 is squeezed in direction A, the first pulley 202 rotates about the pivot 180 in direction B and the second and third pulleys 204, 206 rotate in direction C causing the rack 212 to move distally in direction D. As the trigger 152 is returned to its original position in direction E, the cable pulley system 201 reverses direction and moves the rack 212 proximally in direction F.

FIG. 37 depicts a perspective view of a shaft 212 portion of the hand assemblies 150, 190, 200 shown in FIGS. 32-36 coupled to a rotatable knob 158 element that is coupled to a slide element 154, according to one embodiment. The rotatable knob 158 is used to rotate the shaft clockwise and counterclockwise in directions G. The slide element 154 (FIGS. 32-36) moves the slidably movable sheath 22 forward (distally) D and backward (proximally) F by the various trigger 152 mechanisms described in connection with FIGS. 32-36. As previously discussed, the sheath 22 is advanced distally D when the trigger 152 (FIGS. 32-36) is moved distally in direction E to conceal the electrode 24 and the sheath 22 is retracted proximally F when the trigger 152 (FIGS. 32-36) is squeezed proximally in direction A to expose the electrode 24. FIG. 38 depicts a perspective view of the rotatable knob 158 element showing a shaft to slide element locking feature 214 and a nozzle 174 portion, according to one embodiment. With reference also to FIGS. 32-37, the locking feature 214 in the rotatable knob 158 locks the shaft 160 to the slide element 154 by way of the detent locking element 184 shown in FIG. 34.

Embodiments with Tube-Pinch Mechanism

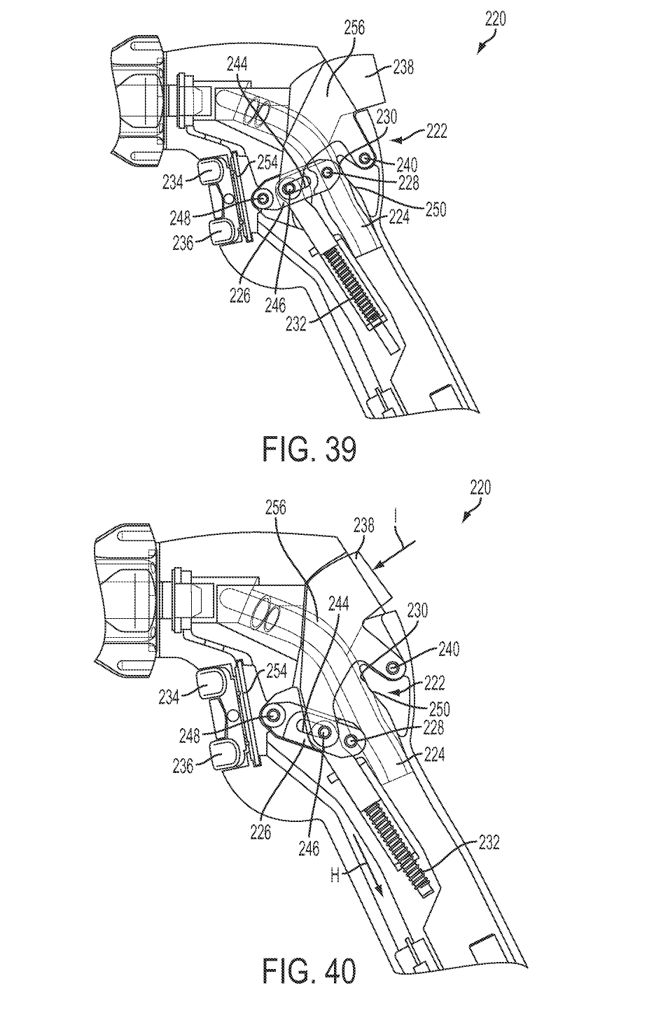

In one embodiment, an electrosurgical device is provided that requires less force to press the irrigation and suction buttons. The device pinches irrigation and suction tubes to close off the flow of suction or water (fluid). In one embodiment, a cam roller is employed to pinch the irrigation and suction tubes shut. The cam roller rests on a cam arm that swings into the tube. As the cam arm swings into the tube it comes parallel to a pinch point on the opposite side of the tube. As the arm becomes parallel it pinches the tube shut. When parallel the arm takes very little force to pinch the tube. The suction and irrigation tubes take little force to pinch close for the first half of closing. Only when the tube is nearly fully shut does it take a lot of force to close. The cam arm takes advantage of this by increasing its mechanical advantage asymptotically the closer it gets to full close. This allows the cam arm to keep the tube pinched with a small spring. It also means the travel to rotate the roller arm off the tube is less. So this configuration uses the same amount of travel as a conventional device to activate the suction or irrigation buttons but with much less force. The electrosurgical device provides less force to fire the suction and irrigation buttons. The cam roller arrangement is compact and simple and requires a smaller spring for easier assembly. These embodiments are described hereinbelow in connection with FIGS. 39-42.

In one embodiment, an electrosurgical device comprises a combination RF monopole, suction, and irrigation device with a tube pinching mechanism comprising a button, cam arm, roller, and spring. In various embodiments, the electro-surgical device may employ RF energy to cauterize tissue in monopolar or bipolar energy modes. In various embodiments, the handle assembly may have a pistol grip configuration, a pencil grip configuration, or may be configured to articulate between a pistol grip and a pencil grip and vice-versa.

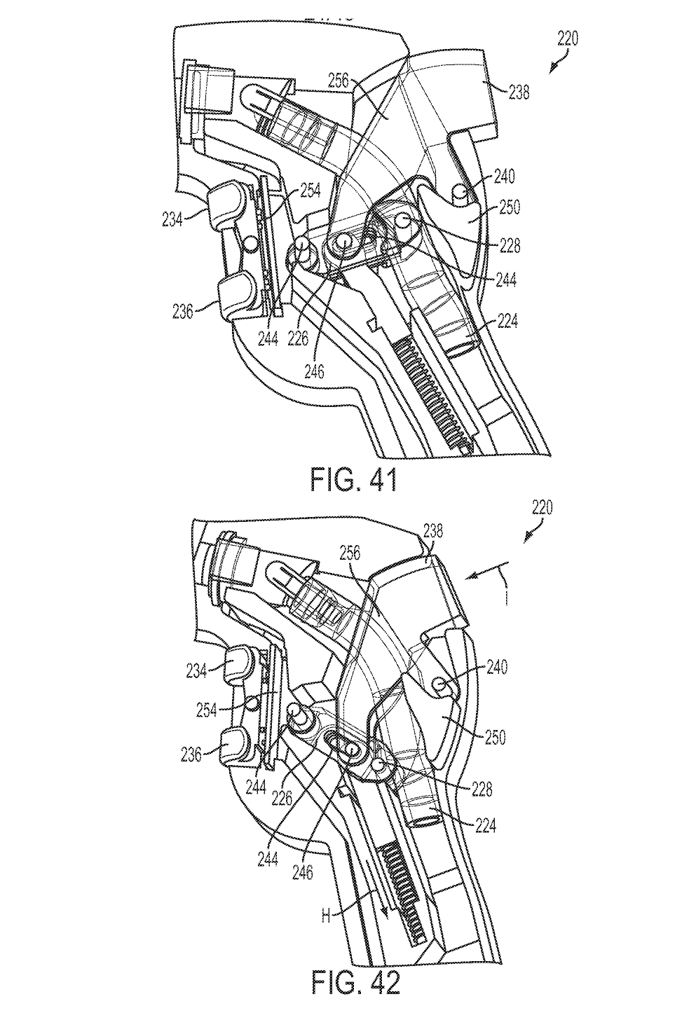

FIG. 39 depicts a transparent side elevational view of a handle assembly 220 of an electrosurgical device comprising a tube pinching mechanism 222 to engage with a tube 224 to deactivate fluid flow or suction, according to one embodiment. FIG. 41 depicts a transparent perspective view of the handle assembly 220 shown in FIG. 39. With reference now to FIGS. 39 and 41, the tube pinching mechanism 222 is engaged with the tube 224 to cut off flow. The tube pinching mechanism 222 comprises a cam arm 226 with a slot 244 defined therein. On one end, the cam arm 226 is pivotally movable about a pivot 248 and comprises a roller 228 on an opposite end. The tube 224 is positioned between the roller 228 and a wall 250 of the handle assembly 220. As the roller 228 moves counterclockwise it pinches the tube 224 against the wall 250 to block flow through the tube 224. A button 238 comprising an arm 256 is used to disengage the roller 228 and open up the flow channel in the tube 224 as shown in FIGS. 40 and 42.

In FIGS. 39 and 41, the button 238 is pushed out by a spring 232 force. A projecting tab 246 is coupled to one end of the arm 256. The tab 246 is confined to move within the slot 244 to cause the cam arm 226 to rotatably move about the pivot 248. The arm 256 also is coupled to a spring 232 which is normally in compression. The spring 232 force holds the button 238 in the outward configuration shown in FIGS. 39 and 41. Thus, when the button 238 is not actuated or pressed, the spring 232 force acts on the arm 238 which rotates about a pivot 240 causes the cam arm 226 to rotate counterclockwise until the roller 246 pinches the tube 224 against the wall 250 at the pinch point 230. The spring 232 force can be selected to accommodate the amount of pinch force that is applied to the tube 224. To release the pinch point 230 and cause fluid to flow within the tube 224, the button 238 is pressed as sown in FIGS. 40 and 42.

FIG. 40 depicts a detailed transparent side elevational view of the handle assembly 220 shown in FIGS. 39 and 41 with the tube pinching mechanism 222 disengaged with the tube 224 to activate fluid flow or suction, according to one embodiment. FIG. 42 depicts a transparent perspective view of the handle assembly shown in FIG. 40 with the tube pinching mechanism disengaged with the tube to activate fluid flow or suction, according to one embodiment. As shown in FIGS. 40 and 42, when the button 238 is pressed as indicated by arrow I, the arm 256 rotates counterclockwise about the pivot 240 causing the projecting tab 246 to engage and slide along the slot 244 within the cam arm 226 and applies a downwardly force. The downwardly force applied by the projecting tab 246 on the cam arm 226 slot 244 causes the cam arm 226 to rotate clockwise and accordingly moves the roller 224 away from the pinch point 230 and thus releases the pinch force applied to the tube 224. The rotation of the arm 256 further compresses the spring 232 in direction H. Accordingly, when the button is released, the spring 232 returns the button 238 to its original position as shown in FIGS. 39 and 41 causing the cam arm 226 to rotate counterclockwise and causing the roller 28 to pinch the tube 224 against the wall 250 at the pinch point 230.

With reference to FIGS. 39-42, the handle assembly 220 further comprises irrigation and suction buttons 234, 236 electrically coupled to circuit board 254. The irrigation and suction buttons 234, 236 are actuated to control the flow and suction through the tube 224 with reduced force.

Embodiments with Articulatable Handle Assembly

In one embodiment, an electrosurgical instrument comprises energy (monopolar or bipolar), suction, and irrigation functions in a device that can be configured either in a pencil or a pistol handle assembly configuration. This expands how the operator (e.g., surgeon) can use the device to access the patient (side versus top). The device can be easily transformed from the pencil to the pistol configuration and vice-versa to provide the operator more options and to stock fewer products on the shelf.

In one embodiment, the electrosurgical device comprises a rotating sealed suction and irrigation chamber with a locking mechanism. The handle assembly may include two components such that it can bend around a pivot. A lock prevents the handle from pivoting until desired. The rotational suction and irrigation chamber or manifold allows the device to transform from straight to bent and vice-versa. The manifold employs an O-ring to keep the joint sealed when articulated. The articulatable device enables a surgeon to utilize the device in two-different modes for better handling in different orientations. Rotating sealed manifold removes the issue of pinch tubing and reduces the force to transform the device.

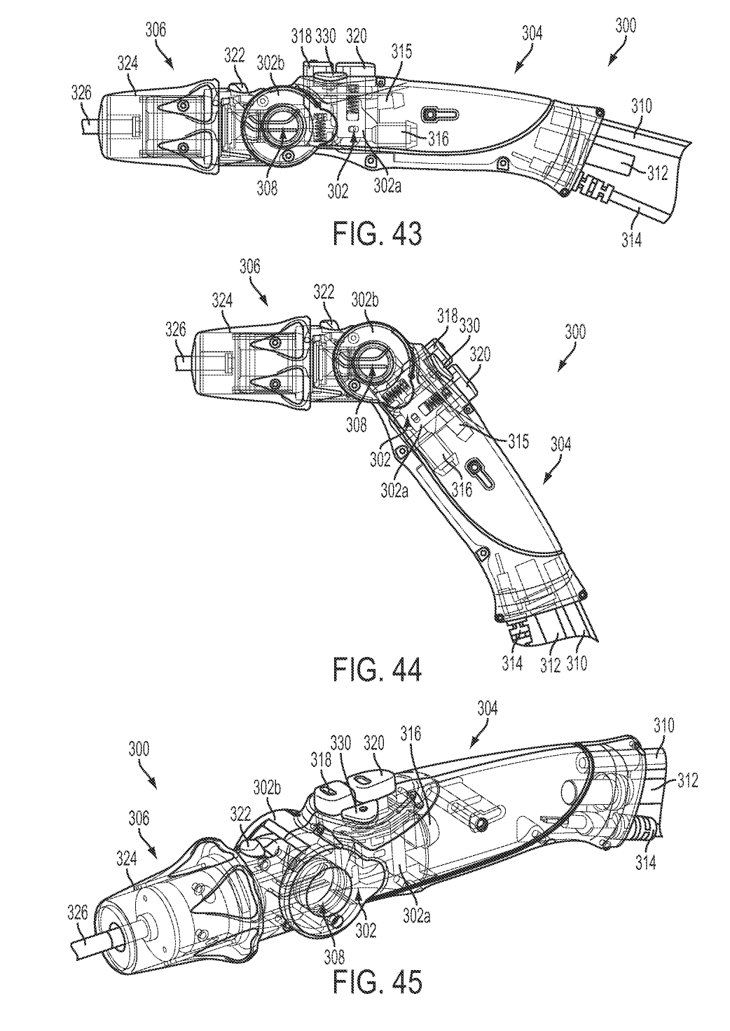

In one embodiment, an electrosurgical device comprises energy (e.g., monopolar or bipolar), irrigation, and suction functions in one combination device that includes a two-part body that can bend and lock into pencil (straight) and pistol (bent) positions. An energy, irrigation, and suction combination device includes a rotational suction and irrigation chamber to allow the body to bend. These embodiments are described hereinbelow in connection with FIGS. 43-74.

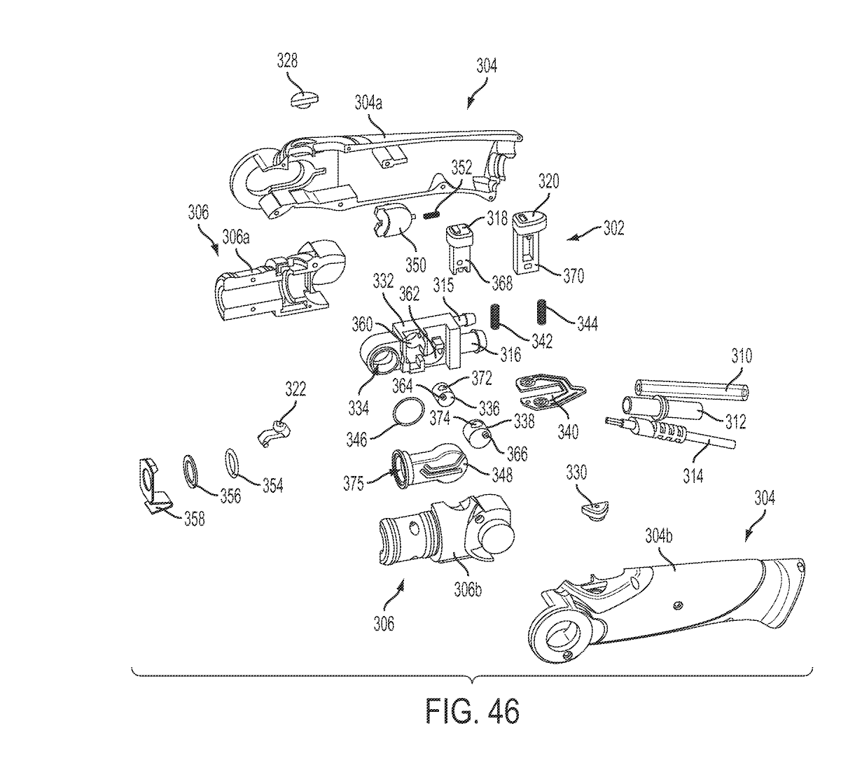

FIG. 43 depicts a side elevational view of a handle assembly 300 of an electrosurgical device comprising an articulating sealed fluid flow manifold assembly 302 with a locking mechanism positioned in a straight/non-rotated (pencil) configuration, according to one embodiment. FIG. 44 depicts a side elevational view of the handle assembly 300 shown in FIG. 43 arranged in a bent/rotated (pistol) configuration, according to one embodiment. FIG. 45 depicts a perspective view of the handle assembly shown in FIGS. 43-44 arranged in a straight non-rotated (pencil) configuration, according to one embodiment. FIG. 46 depicts an exploded view of the handle assembly 300 shown in FIGS. 43-45, according to one embodiment.

With reference now to FIGS. 43-46, in one embodiment the handle assembly 300 comprises a proximal portion 304 and a distal portion 306. The articulating sealed fluid flow manifold assembly 302 comprises a proximal articulating end 302a and a distal articulating end 302b. The proximal portion 306 comprises a right shroud 304a and a left shroud 304b. The distal portion 306 comprises a right shroud 306a and a left shroud 306b. The proximal portion 304 and the distal portion 306 of the handle assembly 300 including the proximal and distal articulating ends 302a, 302b of the manifold assembly 302 articulate at articulation joint 308. A locking element 322 enables the handle assembly 300 to be locked in a predetermined position. The locking element 322 cooperates with an articulation lock 350 and spring 352.

The proximal articulating end 302a of the articulating sealed fluid flow manifold 302 comprises an irrigation port 315 and a suction port 316. An irrigation tube 310, suction tube 312, and an electrical cable 314 are received at a bottom portion of the proximal handle portion 304. The irrigation tube 310 is fluidically coupled to irrigation port 315 and the suction tube 312 is fluidically coupled to the suction port 316. An irrigation button 318 controls the irrigation flow through the irrigation flow path and a suction button 320 controls the suction flow through the suction path. The articulating sealed fluid flow manifold 302 comprises a valve manifold 332 comprising valve apertures 360, 362 configured to rotatably receive an irrigation valve 336 and a suction valve 338 therein. The irrigation valve 336 comprises a projecting tab 364 configured to engage a slot 368 formed on a linear arm portion of the irrigation button 318. The slot 368 acts on the projecting tab 364 to rotate the irrigation valve 336 port 372 to regulate the flow through the irrigation flow path. The suction valve 338 comprises a projecting tab 366 configured to engage a slot 370 formed on a linear arm portion of the suction button 320. The slot 370 acts on the projecting tab 366 to rotate the suction valve 338 port 374 to regulate the flow through the suction flow path. Springs 342, 344 are inserted into the irrigation and suction buttons 318, 320 to return them to their original position. An articulating output manifold 348 is fluidically and rotatably coupled to a main flow port 334 defined by the valve manifold 332. O-ring seal 354 seals the main flow port 375 of the articulating output manifold 348. A washer 356 is located over the O-ring 354 and attached to the main flow port 375 of the articulating output manifold 348. The retainer/electrical contact spring 358 is placed over the washer 356.

A rotatable knob 324 is provided on the distal portion 306 of the handle assembly 300 to manually control the rotation of a shaft 326. Energy buttons 328, 330 are located on the proximal portion 304 of the handle assembly 300. The energy buttons 328, 330 are electrically coupled to a circuit board 340, which electrically coupled to the electrical cable 314. The electrical cable 314 is electrically coupled to an energy source. The circuit board also is electrically coupled to a retainer/electrical spring contact 358, which is electrically coupled to the distal electrode. The energy buttons 328, 330 are utilized to energize the distal electrode through the retainer/electrical spring contact 358, as discussed throughout this disclosure.

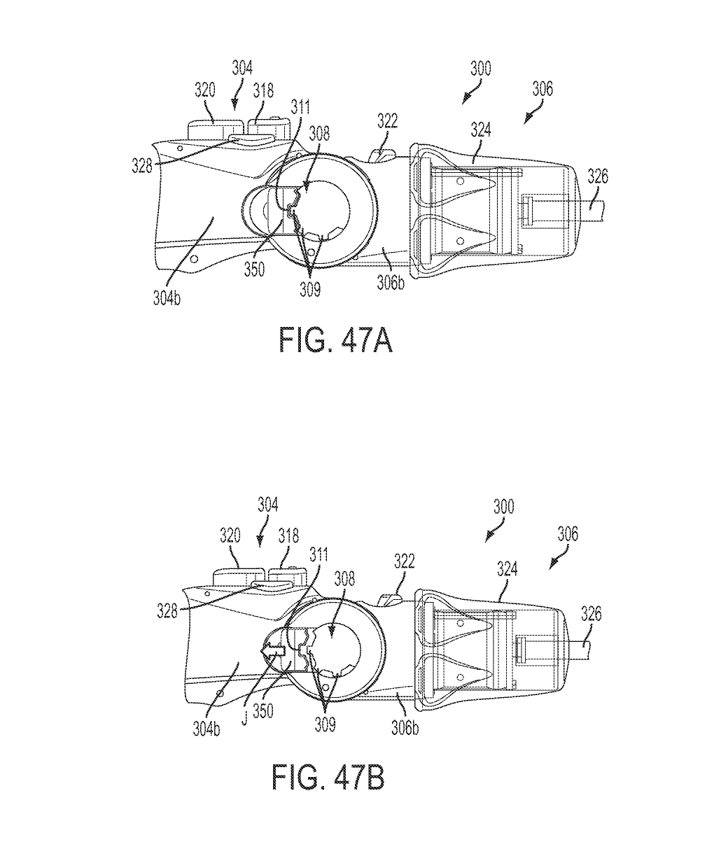

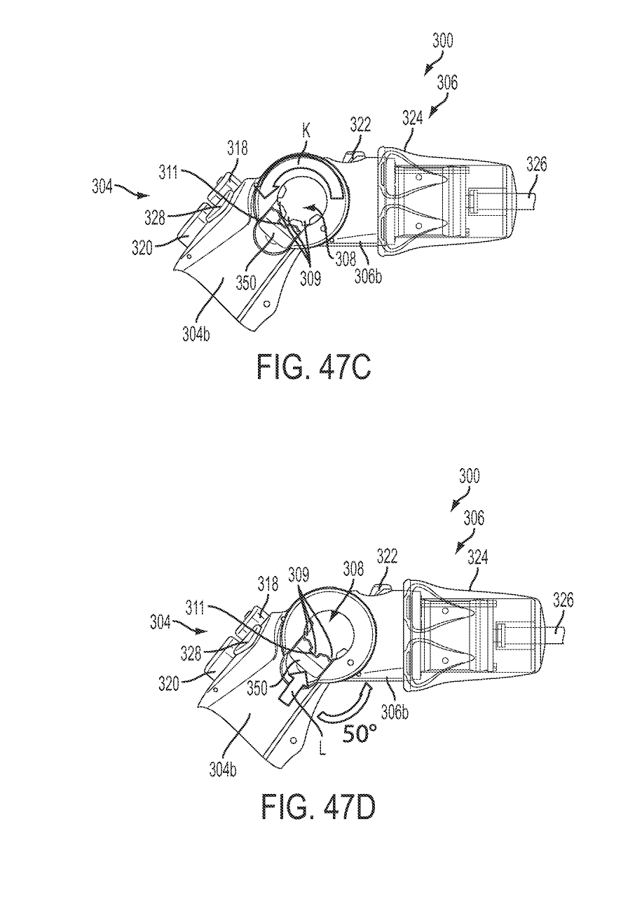

FIGS. 47A-D depict a sequence of steps for rotating and locking the handle assembly 300 shown in FIGS. 43-46, according to one embodiment. In FIG. 47A the proximal and distal handle portions 304, 306 of the handle assembly 300 are in a locked position. The articulating joint 308 comprises a detent element comprising a plurality of detents 309 that engage a slot 311 formed in the spring 352 loaded articulation lock 350. To rotate the proximal handle portion 304, the spring 352 loaded articulation lock 350 is pulled in the direction indicated by arrow J in FIG. 47B to disengage the slot 311 of the detent element from the detent 309 and allowing the proximal handle portion 304 to rotate in the direction indicated by arrow K as shown in FIG. 47C. Once the proximal handle portion 304 is in the desired position, the spring 352 loaded articulation lock 350 is released allowing it to engage with another detent 309 to lock the handle assembly 300 in place.

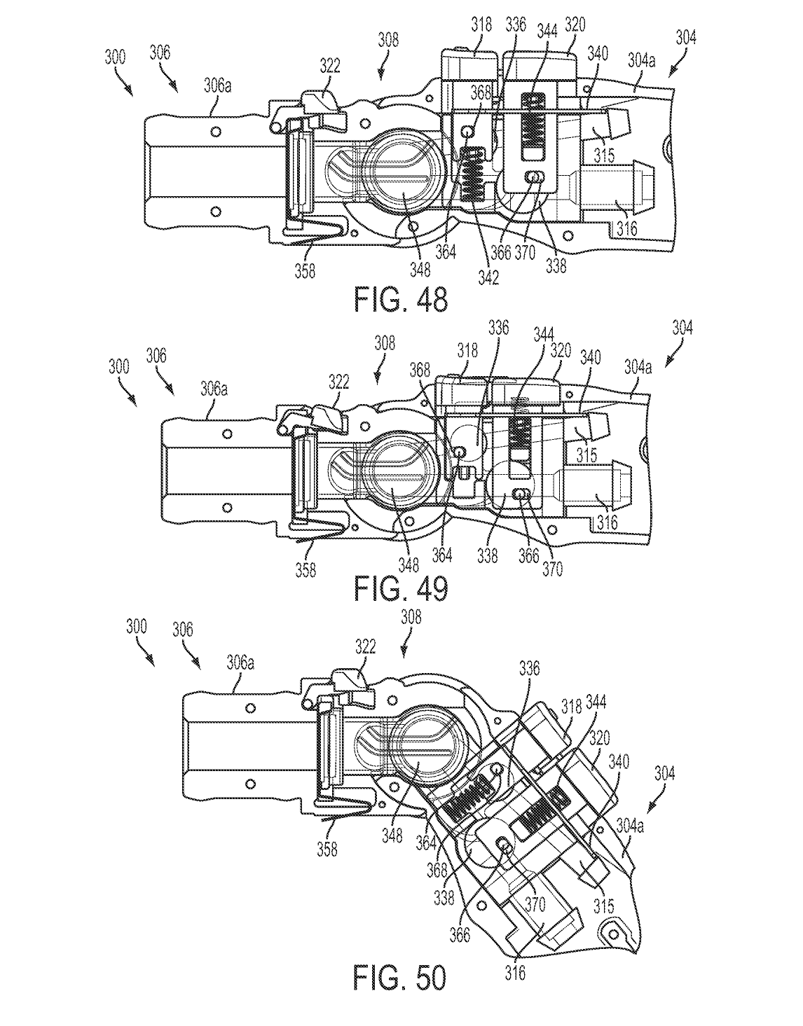

FIG. 48 depicts a partial sectional view of the handle assembly 300 shown in FIGS. 43-46 arranged in a flow blocked straight non-rotated (pencil) configuration, according to one embodiment. The irrigation and suction buttons 318, 320 are in a flow blocked arrangement as indicated by the outward positions of both buttons 318, 320. The springs 342, 344 bias the buttons outwardly to maintain the valves 336, 338 in a closed orientation. The slots 368, 370 in the arms of the buttons 318, 320 engage the projecting tabs 364, 366 to control the rotation of the valves 336, 338.

FIG. 49 depicts a partial sectional view of the handle assembly 300 shown in FIGS. 43-46 arranged in a flow opened straight non-rotated (pencil) configuration, according to one embodiment. As shown, both the irrigation button 318 and the suction button 320 are depressed and the valves 336, 338 are rotated to an open flow position by the slots 368, 370 acting on the projecting tabs 364, 366. The buttons 318, 320 travel over a predetermined distance. In one embodiment, the buttons 318, 320 travel over a predetermined distance of about 0.155 inches.

FIG. 50 depicts a partial sectional view of the handle assembly 300 shown in FIGS. 43-46 arranged in a flow blocked bent rotated (pistol) configuration, according to one embodiment. The proximal handle portion 304 is rotated about the articulation joint 308. The irrigation and suction buttons 318, 320 are now biased by the springs 342, 344 in an outward position to close the flow valves 336, 338.

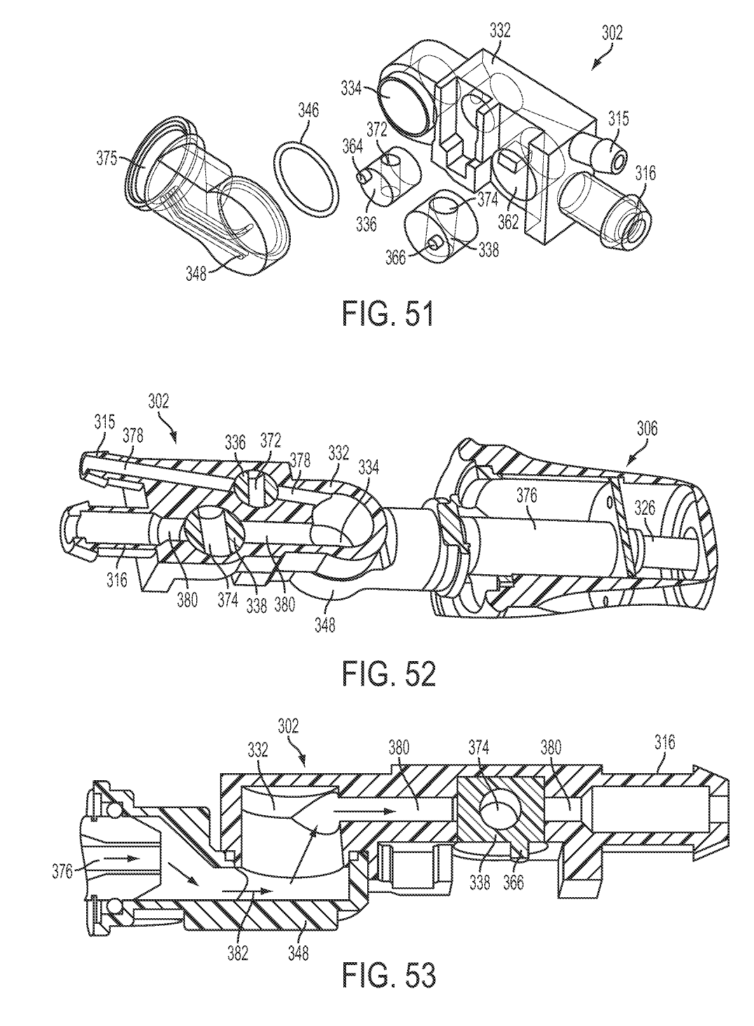

FIG. 51 depicts an exploded view of the articulating sealed fluid flow manifold assembly 302 for the handle assembly 300 shown in FIGS. 43-46 showing the flow paths, stop cock valves 336, 338, and O-ring 346 seal between articulating manifold ends, according to one embodiment. FIG. 52 depicts a perspective sectional view of the articulating sealed fluid flow manifold assembly 302 for the handle assembly 300 shown in FIGS. 43-46 showing the stop cock valves 336, 338 in a closed position to stop the fluid flow in the flow channels 378, 380 of the valve manifold 332, according to one embodiment. FIG. 53 depicts a sectional view of the articulating sealed fluid flow manifold assembly 302 for the handle assembly 300 shown in FIGS. 43-46 showing one of the flow paths 382 before the closed stop clock valve 336, according to one embodiment. In the example illustrated in FIG. 53, the flow path 382 is a suction flow path.

With reference now to FIGS. 51-53, the articulating output manifold 348 is sealed to the main flow port 334 of the valve manifold 332 by O-ring 346. The irrigation and suction valves 336, 338 are inserted into corresponding apertures 360, 362 formed in the valve manifold 332. Irrigation and suction ports 315, 316 fluidically couple to corresponding flow channels 378, 380 within the valve manifold 332. Both flow channels 378, 380 combine at the main flow port 334, which is coupled to the nozzle 376 for delivering and suctioning fluid from the surgical site through the shaft 326.

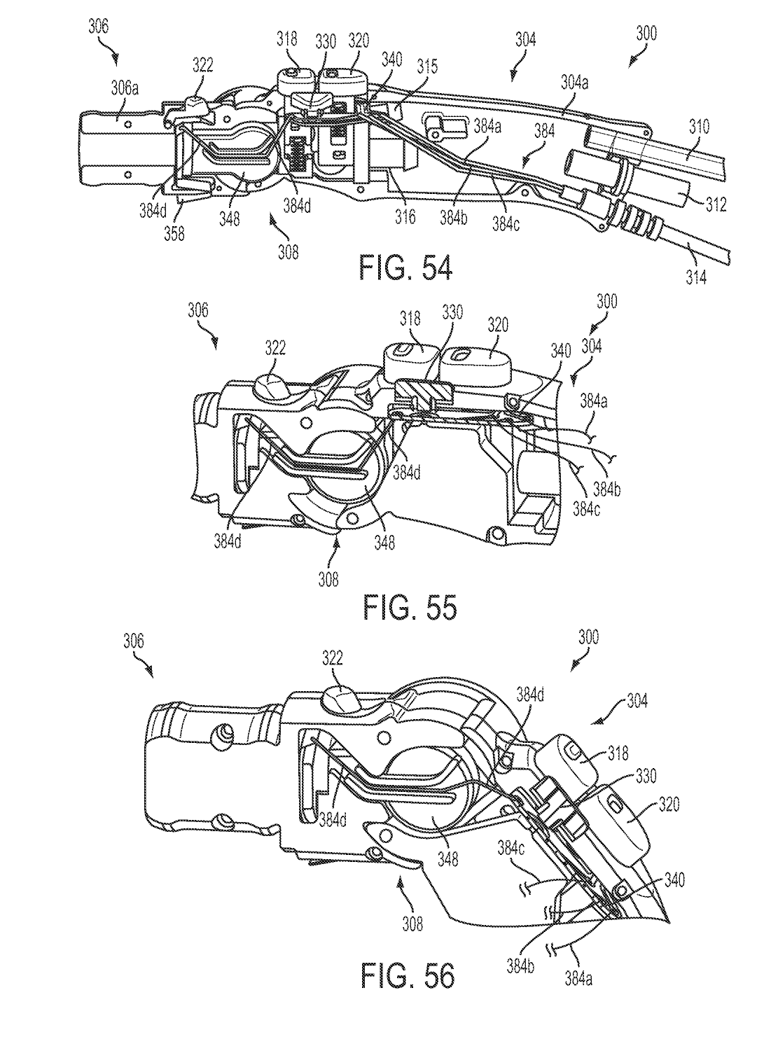

FIG. 54 depicts a sectional view of the articulating sealed fluid flow manifold assembly 302 for the handle assembly 300 shown in FIGS. 43-46 showing an electrical wire path 384, according to one embodiment. FIG. 55 depicts a sectional view of the articulating sealed fluid flow manifold assembly 302 for the handle assembly 300 shown in FIG. 54 arranged in a straight non-rotated (pencil) configuration showing an electrical wire path 384 arranged, according to one embodiment. FIG. 56 depicts a sectional view of the articulating sealed fluid flow manifold assembly 302 for the handle assembly 300 shown in FIG. 54 arranged in a bent rotated (pistol) configuration showing an electrical wire path 384, according to one embodiment.

With reference now to FIGS. 54-56, the wire path 384 includes multiple electrical conductors. In the illustrated embodiment, the wire path 384 includes three electrical conductors 384a-384c. The electrical cable 314 is received at one end of the right shroud 304a and the three individual electrical conductors 384a, 384b, 384c are electrically coupled to the circuit board 340 to couple to the energy source (not shown). An additional electrical conductor 384d is coupled between the circuit board 340 to the retainer/electrical contact spring 358 which is electrically coupled to the electrode at the distal end of the shaft.

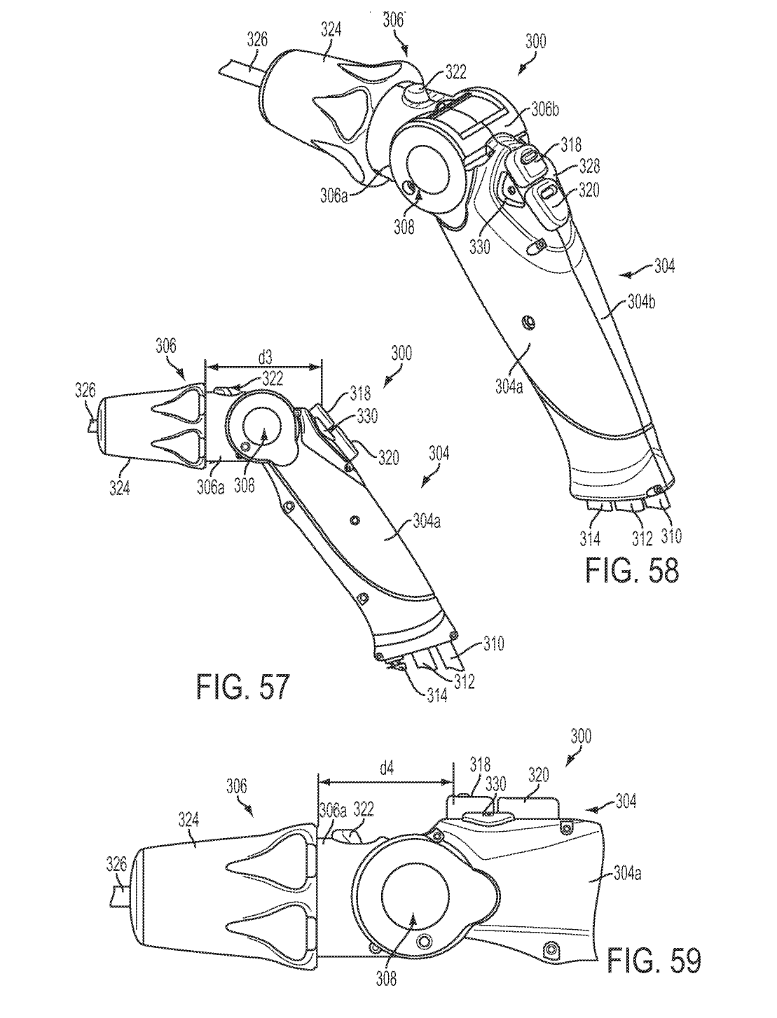

FIGS. 57-58 depict the irrigation and suction buttons 318, 320 layout relative to the rotatable knob 300 for the hand assembly 300 shown in FIGS. 43-46. FIG. 57 depicts a side elevational view of the handle assembly 300 shown in FIGS. 43-46 arranged in a bent rotated (pistol) configuration showing the distance “d3” between the distal button 318 and the distal rotatable knob 324, according to one embodiment. FIG. 58 is a perspective view of the handle assembly 300 shown in FIG. 57 arranged in a bent rotated (pistol) configuration showing the relative distance between the distal button 318 and the rotatable knob 324, according to one embodiment. FIG. 59 depicts a side elevational view of the handle assembly 300 shown in FIGS. 43-46 arranged in a straight non-rotated (pencil) configuration showing the distance “d4” between a distal button 318 and the rotatable knob 324, according to one embodiment. In various embodiments, the distances “d3” and “d4” can be selected such that the handle assembly 300 can be operated with one hand. In one embodiment, the distance “d3” is about 1.85 inches and the distance “d4” is about 1.20 inches.

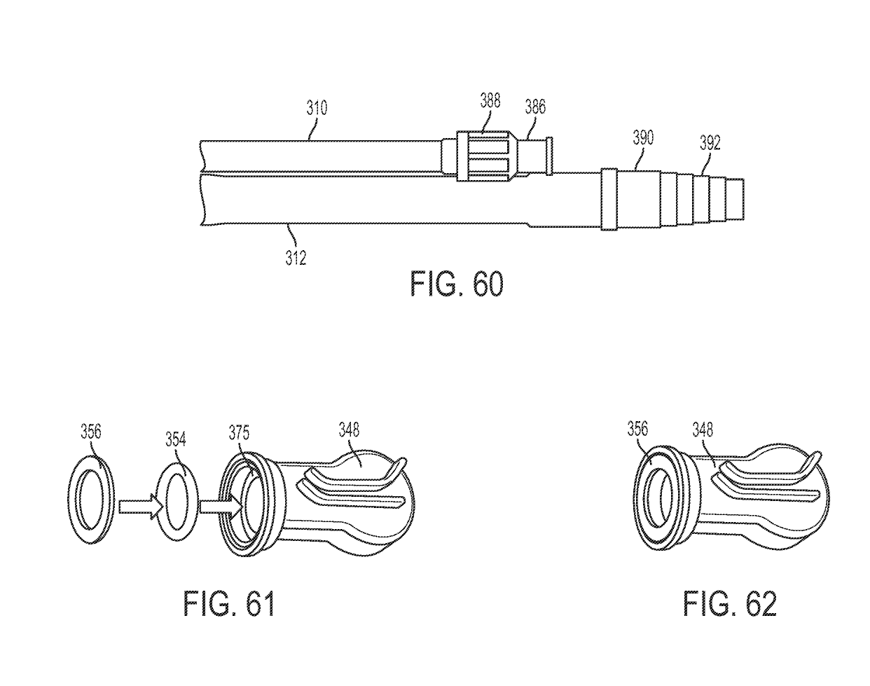

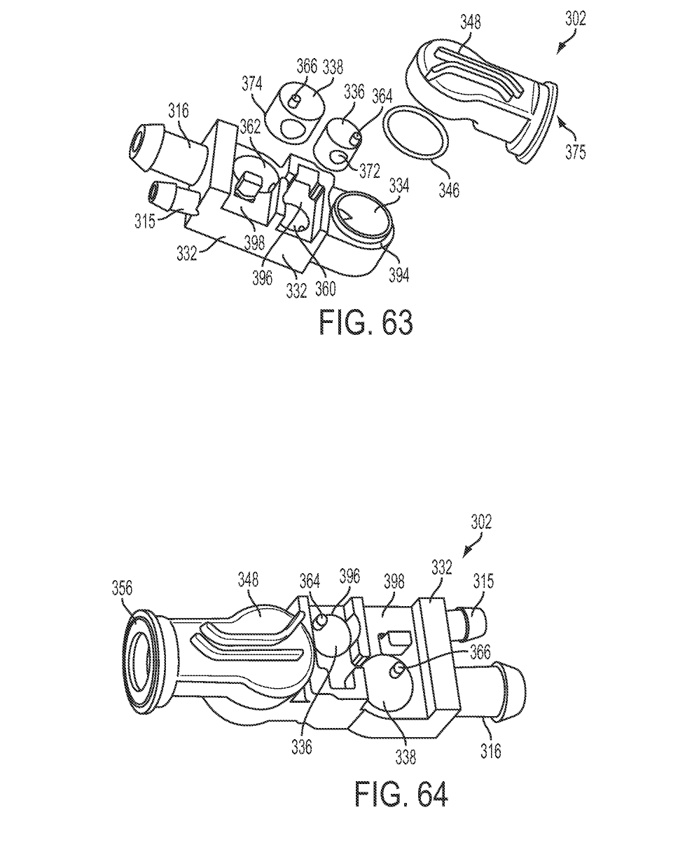

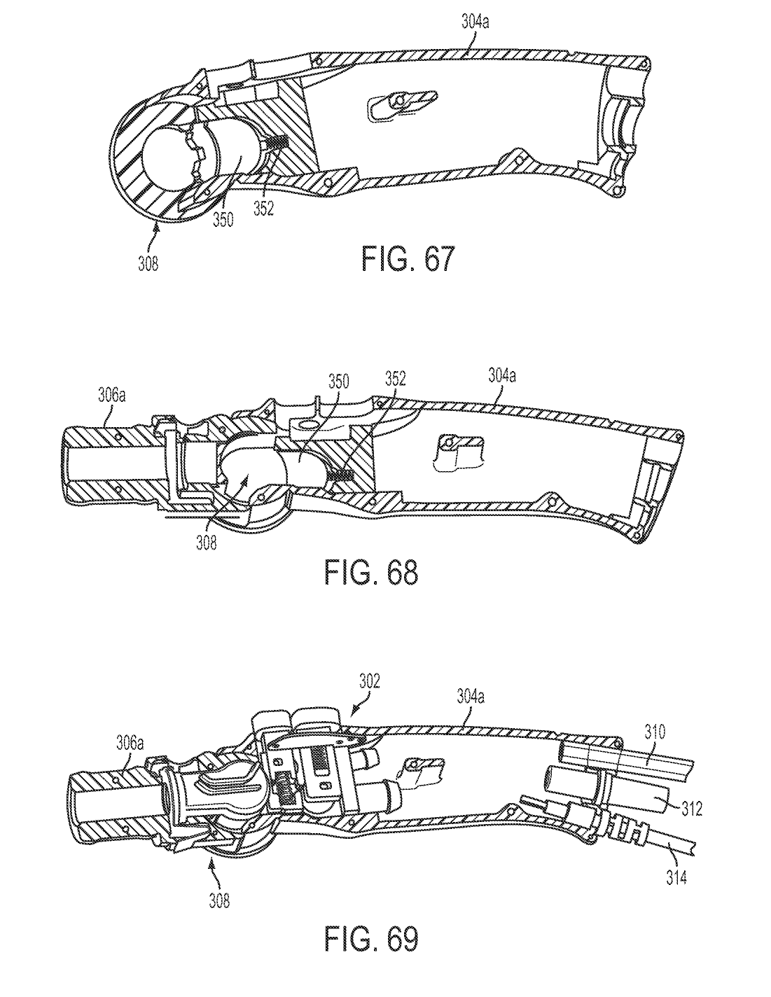

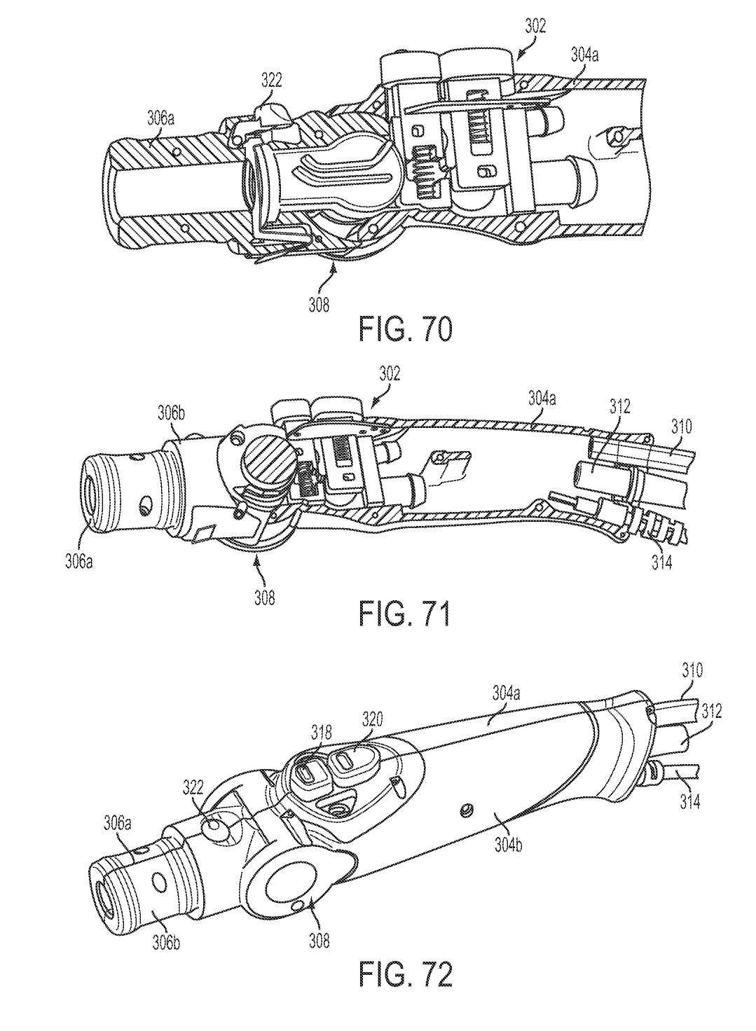

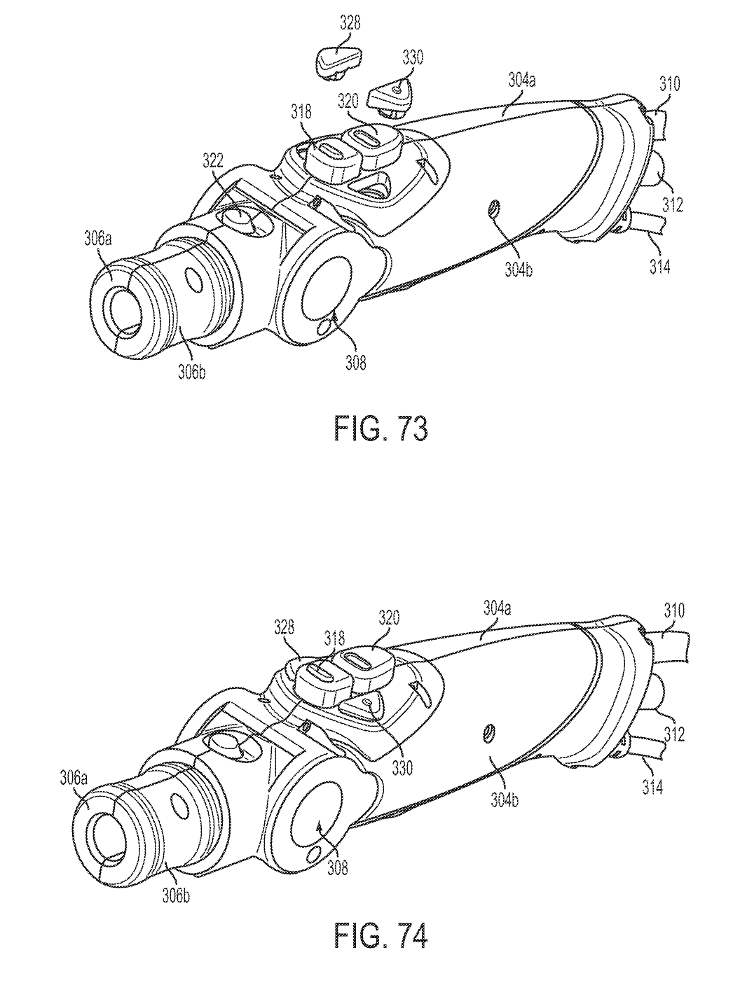

FIGS. 60-74 depict processes for assembling the handle assembly 300. In FIG. 60 the irrigation and suction tubing 310, 312 are attached to flow components and in FIGS. 61-62 a seal is inserted at the main flow port 375 of the articulating output manifold 348. In FIGS. 63-66 the articulating output manifold 348 is assembled to the valve manifold 332. In FIGS. 67-68 the articulation lock 350 and spring 352 is inserted into the articulation joint 308 of the right shroud 304a, 306a portion of the handle assembly 300. In FIG. 69, the manifold assembly shown in FIG. 66 is inserted into the right shroud 304a, 306a portion of the handle assembly 300. In FIG. 70 the unlock arm 322 is inserted. In FIG. 71 the distal left shroud 306b is inserted. In FIG. 72 the proximal left shroud 304b is inserted. In FIG. 73 the energy buttons 328, 330 are inserted, and in FIG. 74 the complete assembled handle assembly 300 is shown. The assembly processes are now described in more detail.

FIG. 60 depict a process for assembling the irrigation and suction tubing 310, 312 and FIGS. 61-62 depict a process for assembling a seal to the main flow port 375 of the articulating output manifold 348. FIG. 60 depicts a process for assembling the irrigation and suction tubes 310, 312 to flow connection components of the manifold subassembly 302 for the handle assembly 300 shown in FIGS. 43-46, according to one embodiment. The irrigation tube 310 is assembled to flow connection components such as an irrigation Luer 386 and irrigation clamp 388. The suction tube 312 is assembled to a suction coupling 390 and suction adapter 392. FIG. 61 depicts a process for assembling the O-ring 354 and washer 356 seal to the main flow port 375 of the articulating output manifold 348 of the handle assembly 300 shown in FIGS. 43-46, according to one embodiment. The O-ring 354 is inserted into the main flow port 375 and then the washer 356 is ultrasonically welded to the output manifold 348. FIG. 62 depicts a perspective view of the assembled articulating output manifold 348 of the handle assembly 300 shown in FIG. 61, according to one embodiment.

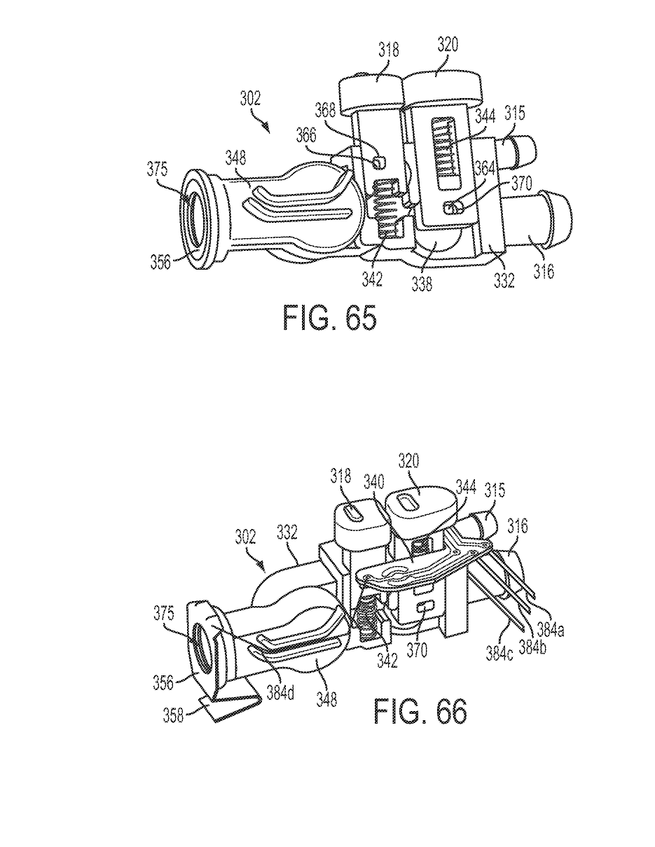

FIGS. 63-66 illustrate a process for assembling the articulating sealed fluid flow manifold assembly 302 portion of the handle assembly 300 shown in FIGS. 43-46. FIG. 63 depicts an exploded view of the manifold subassembly 302 comprising a valve manifold 332 and an output manifold 348. The O-ring 346 is inserted over an edge 394 of the valve manifold 332 about the main flow port 334 and the output manifold 348 is placed over the main flow port 334 of the valve manifold 332. The manifold assembly 302 articulates where the output manifold 348 and the valve manifold 332 components are rotatably connected. Then, the irrigation and suction valves 336, 338 are inserted into the corresponding apertures 360, 362 formed in the body of the valve manifold 332. FIG. 64 depicts a perspective view of the assembled manifold subassembly 302 shown in FIG. 63, according to one embodiment. FIG. 65 depicts a perspective view of the assembled manifold subassembly 302 shown in FIG. 64 with irrigation and suction buttons 318, 320 and corresponding springs 342, 344 inserted in corresponding slots 396, 398 formed on the valve manifold 332 body, according to one embodiment. FIG. 66 depicts a perspective view of the manifold subassembly 302 with irrigation and suction buttons 318, 320 and corresponding springs 342, 344 coupled thereto as shown in FIG. 65 with the circuit board 340 and contact spring 358 attached thereto, according to one embodiment.

FIG. 67 depicts the articulation lock 250 and spring 352 inserted at the articulation joint 308 of the proximal right shroud 304a portion of the handle assembly 300 shown in FIGS. 43-46 with, according to one embodiment.

FIG. 68 depicts the distal right shroud 306a portion of the handle assembly 300 shown in FIGS. 43-46 coupled to the proximal right shroud 304a portion of the handle assembly 300 shown in FIG. 67, according to one embodiment.

FIG. 69 depicts the valve manifold assembly 302 shown in FIG. 66 inserted into the right shroud 304a, 306a portion of the handle assembly 300 shown in FIG. 68, according to one embodiment.

FIG. 70 depicts the unlock arm 322 inserted into the distal right shroud 306a portion of the handle assembly 300 shown in FIG. 69, according to one embodiment.

FIG. 71 depicts the distal left shroud 306b inserted over the distal right shroud 306a portion of the handle assembly 300 shown in FIG. 70, according to one embodiment.

FIG. 72 depicts the proximal left shroud 304b portion of the handle assembly 300 inserted over the handle assembly 300 shown in FIG. 71, according to one embodiment.

FIG. 73 depicts an exploded view of the handle assembly 300 shown in FIG. 72 and energy buttons 328, 330, according to one embodiment.

FIG. 74 depicts a complete handle assembly 300 of the electrosurgical device, according to one embodiment.

Embodiments with Tripper Lockout

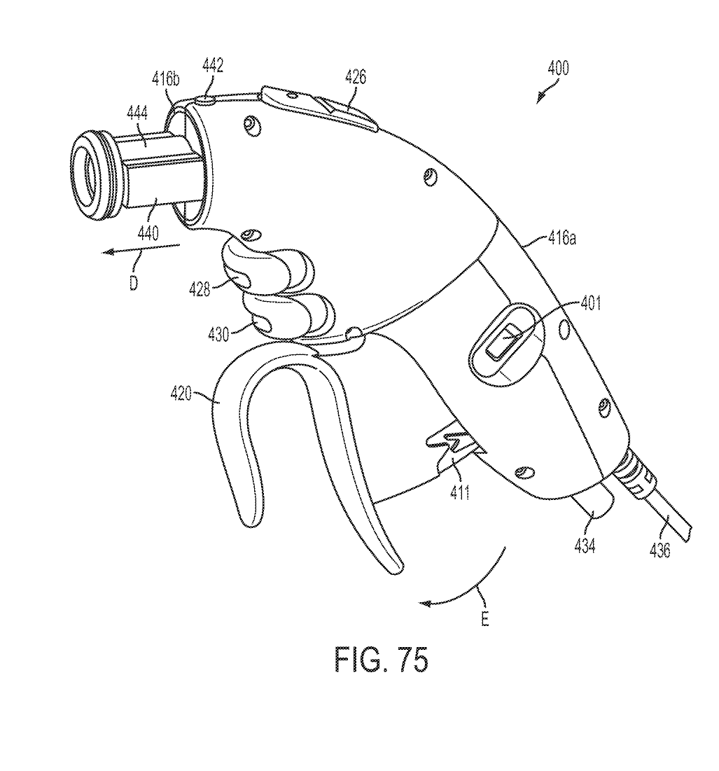

FIG. 75 depicts a perspective view of a handle assembly 400 with a trigger locking mechanism, according to one embodiment, the handle assembly being compatible with the electrosurgical device shown in FIG. 1, according to one embodiment. The handle assembly 400 comprises a trigger 420 having a trigger lock cam 411 formed therein to engage an element of a slidable lockout button 401. In other aspects, the handle assembly 400 includes functional elements similar in structure and operation as the handle assembly 12 described herein with reference to FIGS. 1-31. The features of the handle assembly 400 that are structurally and functionally similar to the handle assembly 12 depicted in FIGS. 1-31 may not be described in the same level of detail for conciseness and clarity of disclosure and, therefore, should not be considered a disclaimer or limitation.

Accordingly, turning now briefly to FIG. 75, the handle assembly 400 comprising a trigger lockout mechanism is compatible with the electrosurgical device 10 shown in FIG. 1, according to one embodiment. The shaft knob of the handle assembly 400 is extended distally along a slide element 440, which encases a nozzle 444 fluidically coupled to a manifold located within the handle assembly 400. As will be described in more detail below, the slide element 440 element is operatively coupled to the trigger 420. The trigger 420 is shown extended in a first “initial” position (e.g., extended distally in direction E) under the influence of a biasing element such as a spring. The position of the trigger 420 shown in FIG. 75 may be referred to herein as the initial or original position in which the trigger 420 is normally configured in and may automatically return to unless a trigger locking mechanism is provided to prevent automatic return to the initial position. The trigger 420 includes a trigger lock cam 411 which cooperates with a pin portion of the trigger lock mechanism. A slidable lockout button 401 is used to disable the lockout.

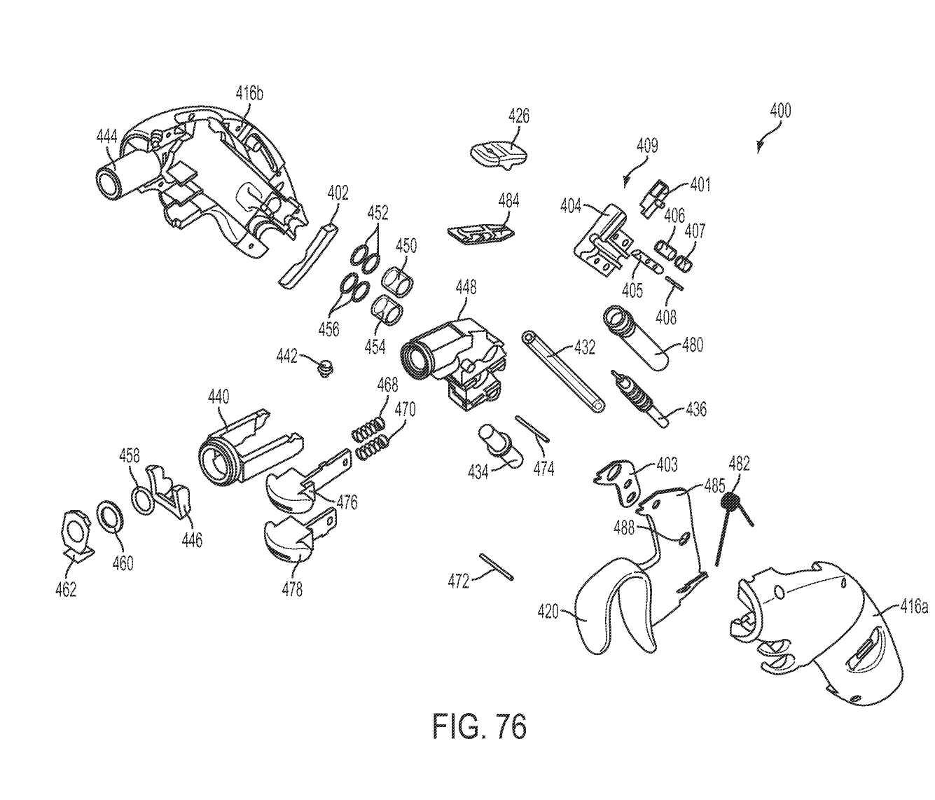

The handle assembly 400 is configured as a pistol grip and comprises left and right handle housings or shrouds 416a, 416b (FIG. 76), a rotatable shaft knob (not shown here), a trigger 420, an energy button 426 or switch, a first flow control button, e.g., an irrigation button 428, and a second flow control button, e.g., a suction button 430. Irrigation and suction tubes 432 (FIG. 76), 434 and an electrical cable 436 enter the handle assembly 400 through a bottom portion. The shaft portion comprises a slidable sheath that extends distally and retracts proximally to respectively conceal and expose the electrode portion. The slidable sheath is operatively coupled to the trigger 420. The irrigation and suction tubes 432, 434 are fluidically coupled to a manifold and respective valves located within the space created between the left and right housing portions 416a, 416b of the handle assembly 400. The irrigation and suction buttons 428, 430 control stopcock valves located within the manifold to control the flow of irrigation fluid through the irrigation tube 432 and suction through the suction tube 434. The electrical cable 436 is electrically coupled to the energy switch 426 and an energy source 8 (FIG. 1). The energy source 8 may be a monopolar or bipolar RF energy source. The energy source 8 may be suitable for therapeutic tissue treatment as well as tissue cauterization/sealing. The energy switch 26 controls the delivery of energy to the electrode. A detailed explanation of each of these control elements is provided hereinbelow. As used throughout this disclosure, a button refers to a switch mechanism for controlling some aspect of a machine or a process. The buttons may be made out of a hard material such as usually plastic or metal. The surface may be formed or shaped to accommodate the human finger or hand, so as to be easily depressed or pushed. Buttons can be most often biased switches, though even many un-biased buttons (due to their physical nature) require a spring to return to their un-pushed state. Terms for the “pushing” of the button, may include press, depress, mash, and punch.

FIG. 76 depicts an exploded view of the handle assembly 400 of the electrosurgical device 10 shown in FIG. 1, according to one embodiment. This view shows the components of the valve manifold located within the handle assembly 400 as well as the elements of a trigger lockout mechanism 409. The trigger lockout mechanism 409 comprises a housing 404 which supports the slidable lockout button 401, a first and second spring 406, 407, a plunger 405, and a pin 408. The left and right shrouds 416a, 416b (handle housings) provide a support for the irrigation and suction fluid flow control buttons 428, 430, the trigger 420, the slide element 440, and the trigger lockout mechanism 409. In addition, the left and right shrouds 416a, 416b also support a valve manifold 448 that is fluidically coupled to the irrigation and suction ports 432, 434 as well as the nozzle 444. The left and right shrouds 416a, 416b also support the energy switch 426 and a circuit board element 84 as well as the electrical cable 436.

In one embodiment, the valve manifold 48 is configured to rotatably support an irrigation valve 450 and a suction valve 454. Although the irrigation and suction valves 450, 454 are stopcock valves, other suitable valves may be employed without departing from the scope of the present disclosure. Irrigation valve O-rings 452 are located about grooves provided on the outer surface of the irrigation valve 450. Suction valve O-rings 456 are located about grooves provided on the outer surface of the suction valve 454. The irrigation and suction valves 450, 454 include tabs or drive dogs that engage respective slots 76, 78 formed on linear arm portions of the respective irrigation and suction buttons 428, 430. The valve manifold 448 also includes an O-ring 458 and a washer 460. A retainer clip 462 that also acts as an electrical spring contact retains the valve manifold 48 to the right shroud 416b. Irrigation and suction ports are used to fluidically couple the valve manifold 448 to the irrigation tube 432 and suction hose 480, which is fluidically coupled to the suction tube coupling 434. The valve manifold 48 is supported within the handle assembly 400 by a pin 474 which is received within corresponding holes formed in each shroud 416a, 416b.

The irrigation and suction buttons 428, 430 (flow control buttons) each include a spring 468, 470 to bias and return the buttons 428, 430 to the distal position after being actuated.

A reversing lever arm 446 is pivotally coupled to the left and right shrouds 416a, 416b via a pin 472. A first projecting tab is received within a corresponding hole in the right shroud 416b. The lever arm 485 includes a pivot hole about which the trigger 420 rotates. The reversing lever arm 446 is operatively coupled to the slide knob 440, which is operatively coupled to a slidable sheath 22 (FIGS. 1, 3, 5, 7) of a shaft portion 14 (FIGS. 1, 3, 5, 7). The notch portion of the lever arm 485 of the trigger 420 engages a projecting tab of the reversing lever arm 446 to cause the motion of the slide knob 440 to match the motion of the trigger 420, e.g., trigger 420 forward—slide knob 440 forward and trigger 420 backward—slide knob 440 backward. The slide knob 440 is slidably movable over the nozzle 444, which is fluidically coupled to the output port of the valve manifold 448. A torsion spring 482 is located over a hub 490 formed in the right shroud 416b. One arm of the torsion spring 482 is coupled to a slot 488 formed in the lever arm 485 portion of the trigger 420 and another arm of the torsion spring 482 is located against a back wall of the right shroud 416b to bias the trigger 420 outwardly in a distal position when not squeezed and to return the trigger 420 outwardly to the distal position when the operator releases the trigger 420.

The circuit board 484 is mounted to the right shroud 416b. The energy switch 426 is electrically coupled to the circuit board 484. The electrical spring contact 462 also is electrically coupled to the circuit board 484. The electrical spring contact 462 is electrically coupled to the electrode 24 (FIGS. 1, 3, 5). The electrical cable 436 couples energy from the energy source to the circuit board 484. When the energy switch 426 is activated energy is coupled from the energy source to the electrode 24 via the electrical spring contact 462.

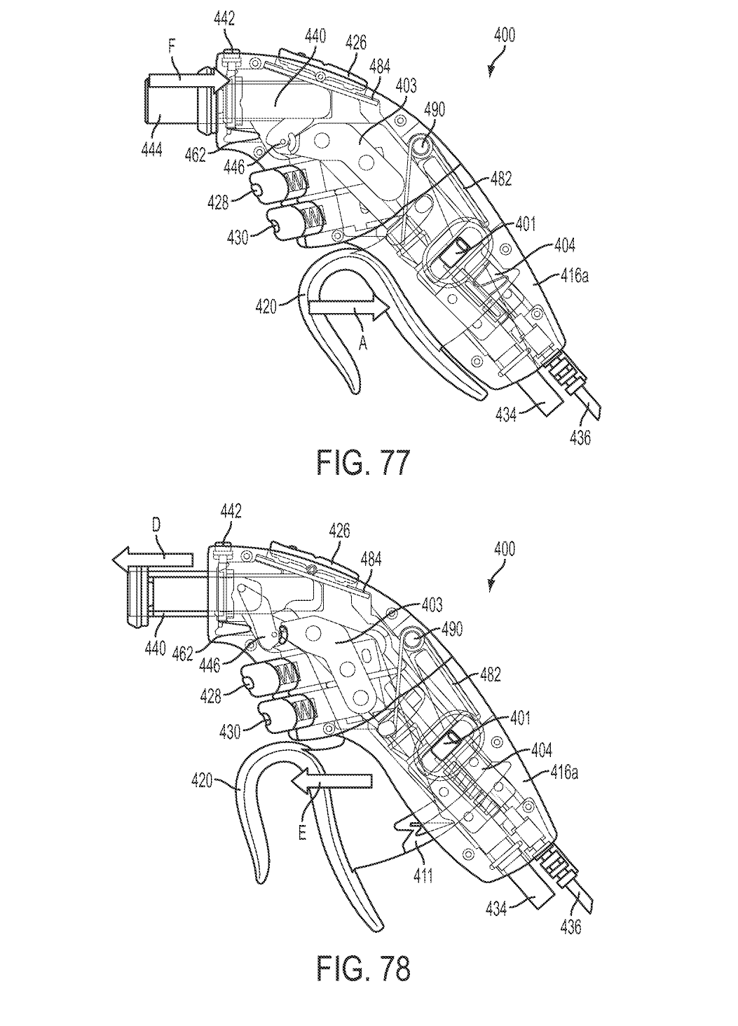

FIG. 77 depicts a partial transparent side elevational view of the handle assembly 400 shown in FIG. 75 with a trigger 420 portion in a second position and a slidably movable element 440 in a retracted position, according to one embodiment. In order to prevent the trigger 420 from locking once squeezed in direction A, the slidable lockout button 401 is slid forward, to enable trigger 420 to be squeezed in direction A without engaging the trigger lockout mechanism. When the trigger 420 is back, as shown, the shaft is back in direction F. This position allows energy to be applied to the tip.

FIG. 78 depicts a partial transparent side elevational view of the handle assembly 400 shown in FIG. 75 with the trigger 420 portion in a first position and the slidably movable element 440 is in an extended position, according to one embodiment. Also shown is the trigger lock cam 411, which engages the pin portion of the trigger lockout mechanism 409. The trigger 420 is sprung forward in direction E to enter the suction and irrigation mode(s). The nozzle 444 moves distally in direction D over the electrode tip. The flow control buttons 428, 430 are shown pressed.

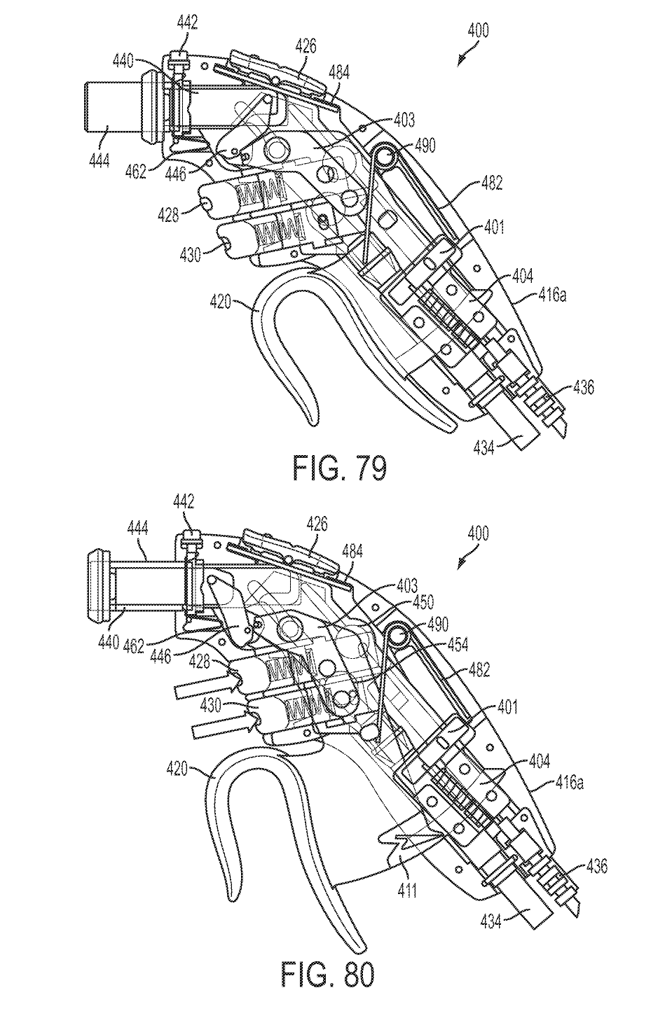

FIG. 79 depicts a partial transparent side elevational view of the handle assembly 400 shown in FIG. 75 with the trigger 420 portion in a second position and the shaft in a retracted position and fluid flow control buttons, e.g., 428, 430, pushed out to disable fluid flow, according to one embodiment. The trigger 420 is shown in a locked position where a distal portion of the slidable element 401 engages a notch portion of the plunger 405.

FIG. 80 depicts a partial transparent side elevational view of the handle assembly 400 shown in FIG. 75 with the trigger 420 portion in a first position and the shaft in an extended position and the fluid flow control buttons 428, 430 pushed in to rotate the flow valves 450, 454 and enable fluid flow, e.g., irrigation and suction, according to one embodiment. The trigger 420 includes a trigger lock cam 411.

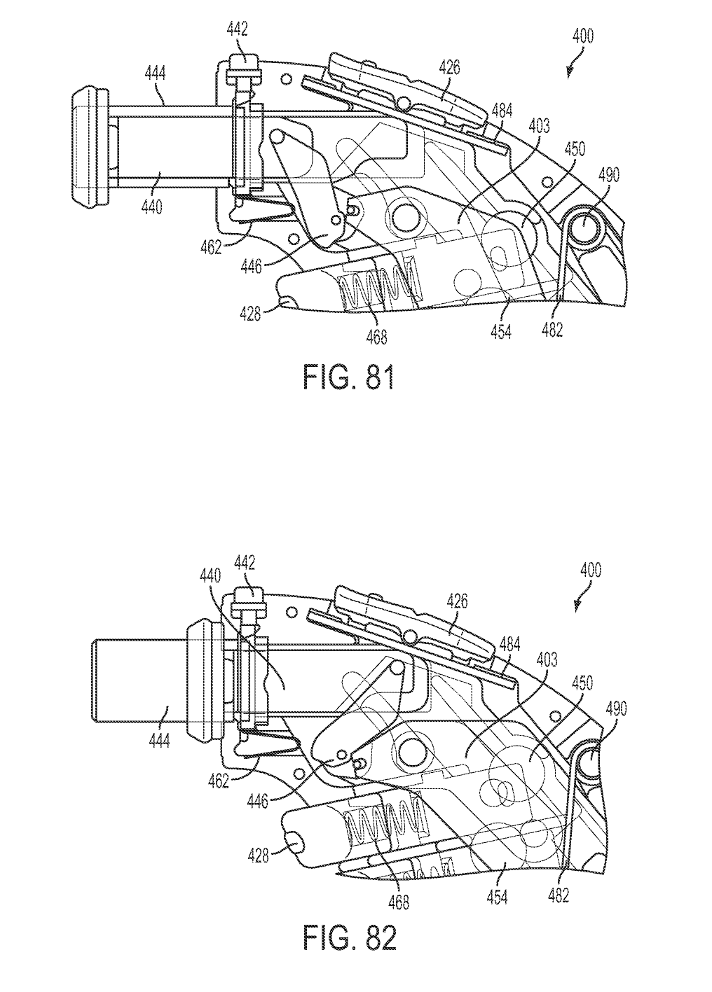

FIG. 81 depicts a partial cut-away transparent side elevational view of the handle assembly 400 shown in FIG. 75 with the reversing arm 446 operatively coupled to the trigger 420 where the trigger 420 is located in a forward (distal) position as biased by a torsion spring 482 to extend the slide element 440 forward in a distal direction to conceal the electrode, according to one embodiment.

FIG. 82 depicts a partial cut-away transparent side elevational view of the handle assembly 400 shown in FIG. 75 with the trigger 420 in a backward (proximal) position squeezed to overcome the bias force of the torsion spring 482 to retract the slide element 440 backward in a proximal direction to expose the electrode, according to one embodiment. The torsion spring 482 returns the trigger 420 to its initial location unless the trigger lockout mechanism 409 is engaged to lock the trigger, according to one embodiment.

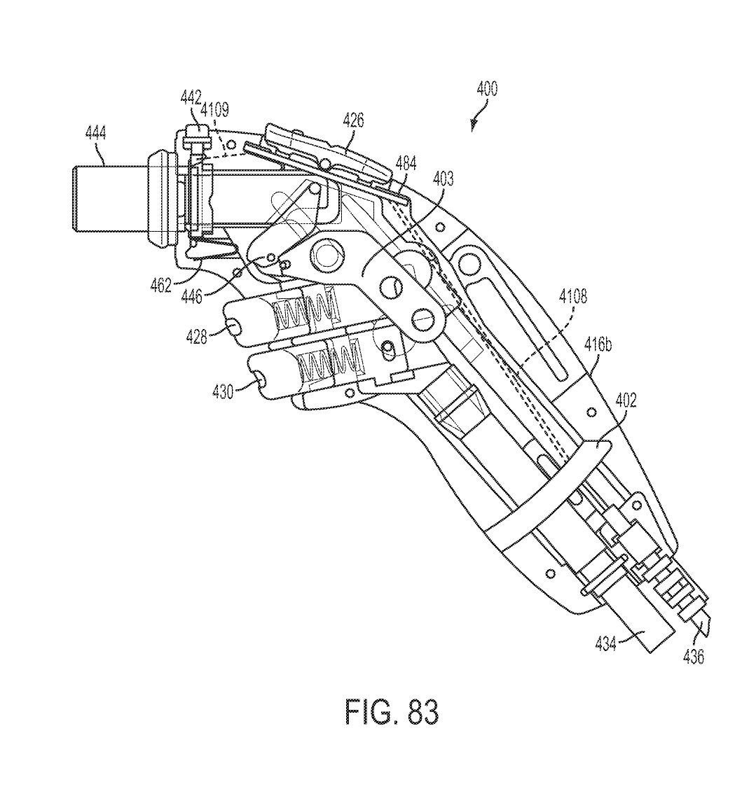

FIG. 83 depicts a partial cut-away transparent side elevational view of the handle assembly 400 shown in FIG. 75 with a wire cover 402, according to one embodiment. The wire cover 402 covers the wires 4108, 4109 along an internal wire path to prevent the trigger 420 from hitting the wires 4108, 4109 during operation.

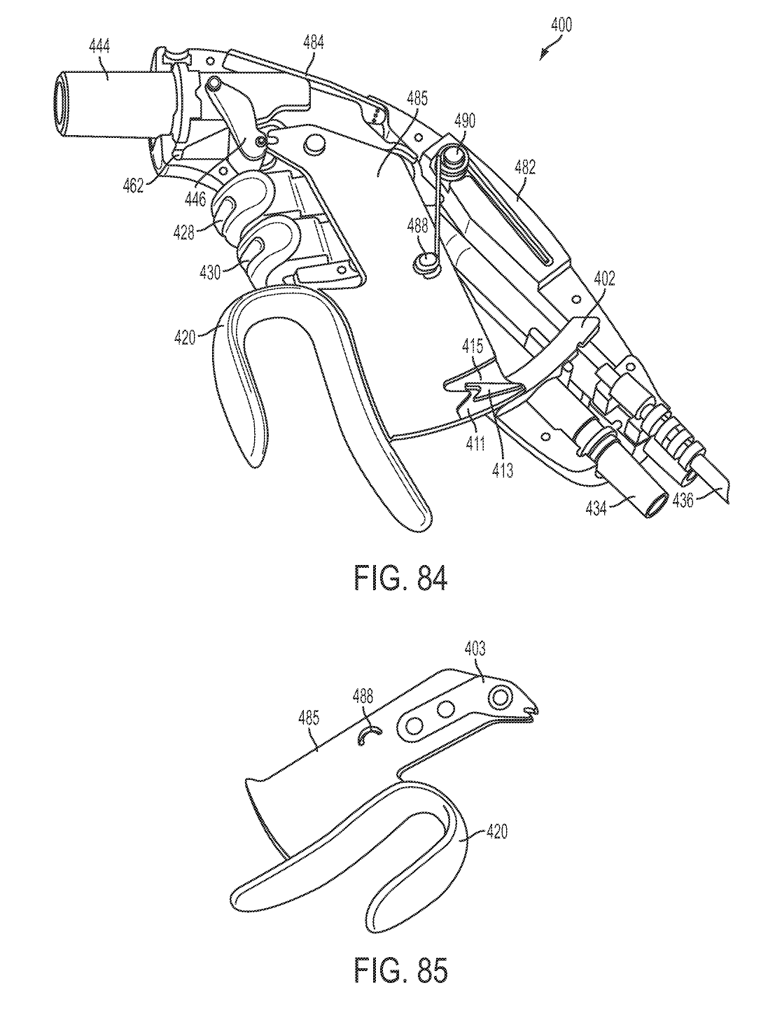

FIG. 84 depicts a partial perspective view of the handle assembly 400 shown in FIG. 75 showing the trigger 420 and reverse arm 446 elements, according to one embodiment. This view also shows the trigger lock cam 411 which comprises a channel 415 formed around a wall 413. The pin 408 portion (FIG. 2) of the trigger lockout mechanism 409 (FIG. 2) is slidable within the channel 415 as guided by the wall 413 to lockout the trigger 420 after it has been squeezed.

FIG. 85 depicts a perspective view of the trigger 420 and the trigger plate 403 elements of the handle assembly 400 shown in FIG. 75, according to one embodiment. The trigger plate 403 engages the reverse arm 446 to enable the trigger 420 motion to match the slidable element 440 motion, e.g., trigger forward—knob forward and trigger back—knob back.

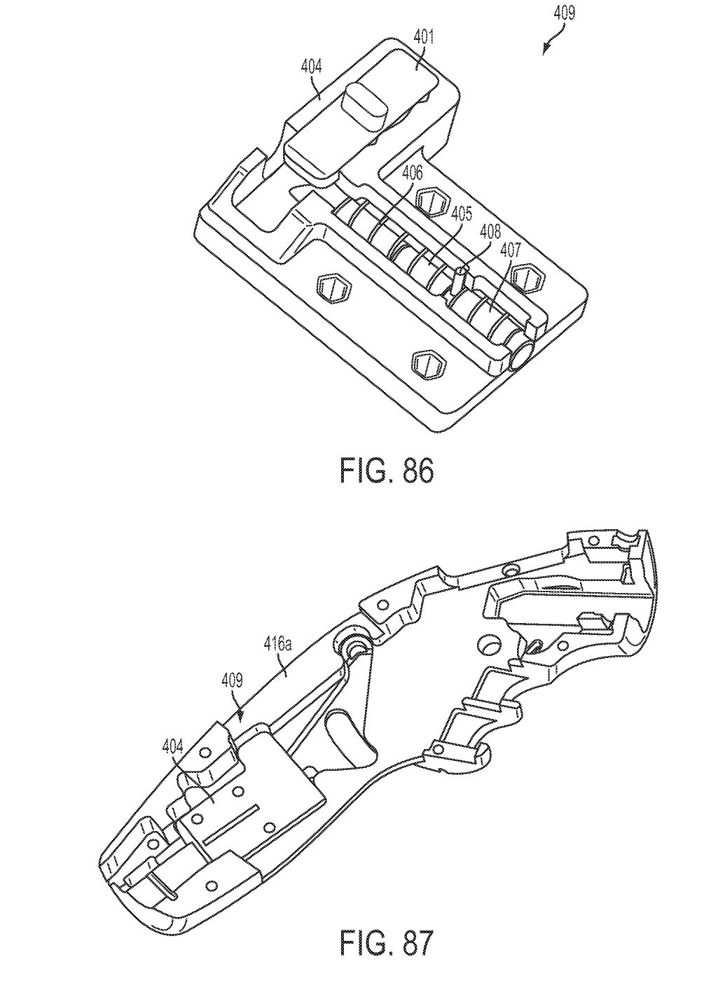

FIG. 86 depicts a perspective view of the trigger lockout assembly 409 for the handle assembly 400 shown in FIG. 75, according to one embodiment. The slidable lockout button 401 is located within the housing 404 (box) along with springs 406, 407, and the plunger 405. The pin 408 is inserted through an aperture formed through the body of the plunger 405. The pin 408 engages the trigger lock cam 411 channel 415 formed around the wall 413.

FIG. 87 depicts perspective view of an interior portion of the left shroud 416a of the handle assembly shown in FIG. 75 showing the trigger lockout assembly 409 located therein, according to one embodiment.

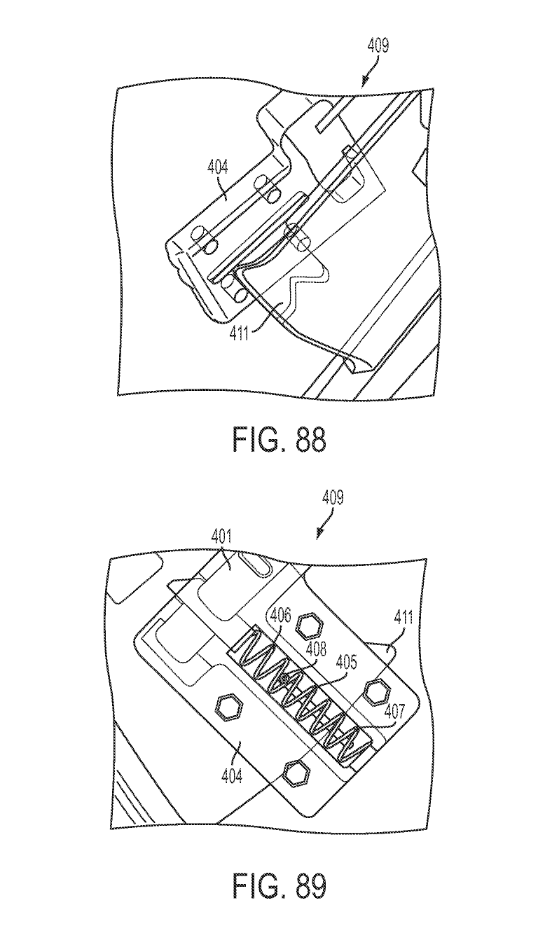

FIG. 88 depicts a perspective view of a housing 404 portion of the trigger lockout assembly 409 shown in FIG. 86, according to one embodiment. The housing 404 portion of the trigger lockout assembly 409 is positioned within the left shroud 416a such that the pin 408 engages the trigger lock cam 411 when the trigger 420 is squeezed.

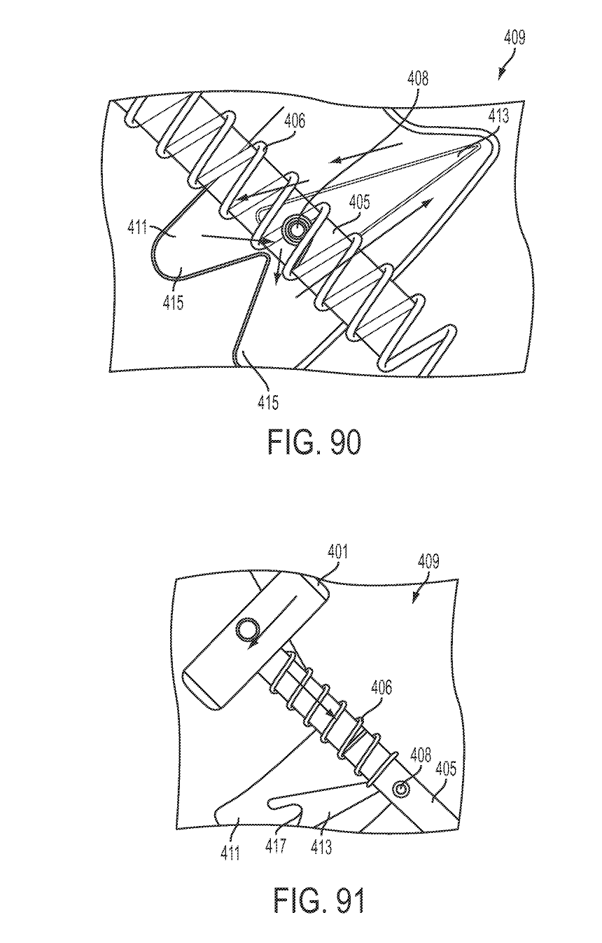

FIG. 89 depicts a perspective view of the trigger lockout assembly 409 shown in FIG. 86, according to embodiment. As shown in FIG. 89, with the slidable lockout button 401 in a first position enables the plunger 405 to move within the spring aperture to enable the pin 408 to engage the trigger lock cam 411 and lockout the trigger 420 once it has been squeezed, as shown in FIG. 90, where the arrows represent the path of the pin 408 moving through the channel 415 of the trigger lock cam 411. To lock the trigger 420, the pin rests in a recess 427 (shown in FIG. 91). When the pin 408 rests in the notch 417, the trigger 420 is locked out. Squeezing the trigger 420 again, however, forces the pin 408 to continue following the contour of the wall 413 to release or unlock the trigger 420 as shown by the arrows.

FIG. 91 depicts the trigger lockout assembly 409 shown in FIG. 86 being bypassed by sliding the slidable lockout button 401, according to one embodiment. As shown by the arrows, sliding the slidable lockout button 401 forward forces the pin 408 down out of the trigger lock cam 411 channel 415 (path) such that the pin 408 does not locate in the notch 417 to lockout the trigger 420.

### Other Example Features

While the examples herein are described mainly in the context of electrosurgical instruments, it should be understood that the teachings herein may be readily applied to a variety of other types of medical instruments. By way of example only, the teachings herein may be readily applied to tissue graspers, tissue retrieval pouch deploying instruments, surgical staplers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The disclosed embodiments have application in conventional endoscopic and open surgical instrumentation as well as application in robotic-assisted surgery.

Embodiments of the devices disclosed herein can be designed to be disposed of after a single use, or they can be designed to be used multiple times. Embodiments may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

It is worthy to note that any reference to “one aspect,” “an aspect,” “one embodiment,” or “an embodiment” means that a particular feature, structure, or characteristic described in connection with the aspect is included in at least one aspect. Thus, appearances of the phrases “in one aspect,” “in an aspect,” “in one embodiment,” or “in an embodiment” in various places throughout the specification are not necessarily all referring to the same aspect. Furthermore, the particular features, structures or

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely examples and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

Some aspects may be described using the expression “coupled” and “connected” along with their derivatives. It should be understood that these terms are not intended as synonyms for each other. For example, some aspects may be described using the term “connected” to indicate that two or more elements are in direct physical or electrical contact with each other. In another example, some aspects may be described using the term “coupled” to indicate that two or more elements are in direct physical or electrical contact. The term “coupled,” however, also may mean that two or more elements are not in direct contact with each other, but yet still co-operate or interact with each other.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

In summary, numerous benefits have been described which result from employing the concepts described herein. The foregoing description of the one or more embodiments has been presented for purposes of illustration and description. It is not intended to be exhaustive or limiting to the precise form disclosed. Modifications or variations are possible in light of the above teachings. The one or more embodiments were chosen and described in order to illustrate principles and practical application to thereby enable one of ordinary skill in the art to utilize the various embodiments and with various modifications as are suited to the particular use contemplated. It is intended that the claims submitted herewith define the overall scope.

Various aspects of the subject matter described herein are set out in the following numbered clauses:

1. An electrosurgical device, comprising: a handle assembly comprising: a valve having an input port and an output port, the input port fluidically coupled to either an irrigation source or a suction source; at least one button operatively coupled to the valve to control flow through the valve; at least one switch to electrically couple energy from an energy source; and a trigger; a shaft comprising: a slidable element operatively coupled to the trigger; and an electrode electrically coupled to the switch; wherein the trigger is operable to position the slidable element relative to the electrode to conceal or expose the electrode.

2. The electrosurgical device of clause 1, comprising a nozzle fluidically coupled to the output port of the valve, wherein the slidable element is fluidically coupled to the nozzle.

3. The electrosurgical device of clause 1, wherein the slidable element comprises a sheath fluidically coupled to the output port of the valve.

4. The electrosurgical device of clause 1, comprising a lever operatively coupled to the trigger and the slidable element such that the motion of the trigger is correlated to the motion of the slidable element.

5. The electrosurgical device of clause 4, wherein the slidable element is retracted in a proximal direction when the trigger is actuated in a proximal direction; and wherein the slidable element is advanced in a distal direction when the trigger is actuated in a distal direction.

6. The electrosurgical device of clause 1, comprising a cam operatively coupled to the trigger and the slidable element such that the motion of the trigger is correlated to the motion of the slidable element.

7. The electrosurgical device of clause 1, comprising a gear assembly operatively coupled to the trigger and the slidable element such that the motion of the trigger is correlated to the motion of the slidable element.

8. The electrosurgical device of clause 1, comprising a cable pull system operatively coupled to the trigger and the slidable element such that the motion of the trigger is correlated to the motion of the slidable element.

9. The electrosurgical device of clause 1, comprising a spring operatively coupled to the trigger to return the trigger to its un-triggered state.

10. The electrosurgical device of clause 1, comprising a valve manifold to contain the valve.

11. The electrosurgical device of clause 1, wherein the button comprises a linear arm portion defining a slot and the valve comprises a projecting tab operatively coupled to the slot, wherein liner motion of the button arm translates into rotation of the valve.

12. The electrosurgical device of clause 1, wherein the valve is a stopcock valve.

13. The electrosurgical device of clause 1, comprising a locking mechanism operatively coupled to the trigger.

14. The electrosurgical device of clause 13, wherein the locking mechanism comprises a plurality of detents to lock the trigger at a plurality of positions.

15. The electrosurgical device of clause 13, wherein the trigger is movable from a first position to a second position and the locking mechanism maintains the trigger locked in the second position.

16. The electrosurgical device of clause 13, wherein the locking mechanism comprises a lever and a spring located on the trigger that automatically latches when the trigger is closed.

17. The electrosurgical device of clause 16, wherein the lever is pushed in an opposite direction from the latching direction to unlock the trigger.

18. The electrosurgical device of clause 16, comprising a spring biased member and a locking cam operatively coupled to the trigger, wherein the locking mechanism locks the trigger when the trigger moved in a first direction and unlocks the trigger when the trigger is moved again in the closed direction.

19. The electrosurgical device of clause 1, wherein the slide element is operatively coupled to a spring to return the slide mechanism to its starting position automatically.

20. An electrosurgical device, comprising: a handle assembly defining a rigid wall; a cam arm supported by the handle assembly and pivotally movable about a first pivot on one side and comprising a roller supported on another side, the cam arm defining a slot therebetween; a first button supported by the handle assembly and pivotally movable about a second pivot, the first button comprising: an arm; and a projecting tab slidably engaged with the slot; a second button pivotally coupled to the cam arm at the first pivot; and a bias element acting on the arm of the first button to force the cam arm to pivotally move in a first direction and the roller to move toward the rigid wall of the handle assembly; wherein pressing the first button overcomes the force of the bias element and the projecting tab applies a force on the slot to pivotally move the cam arm in a second direction and to move the roller away from the rigid wall of the handle assembly.

21. The electrosurgical device of clause 20, comprising a flexible tube located between the rigid wall and the roller such that the roller applies a pinching force against the tube when the cam arm is pivotally forced in the first direction by the bias element.

22. The electrosurgical device of clause 21, wherein the pinching force against the tube is removed when the cam arm is pivotally moved in the second direction.

23. The electrosurgical device of clause 20, wherein the second button is operable to partially control the force applied to the cam arm.

24. An electrosurgical device comprising: a handle assembly comprising an articulating handle comprising a proximal housing and a distal housing rotatably coupled at an articulation joint; a sealed fluid flow manifold assembly configured to articulate about the articulation joint; and a locking mechanism positioned at the articulation joint to lock the proximal housing and the distal hosing in a configuration.

25. The electrosurgical device of clause 24, wherein the articulating fluid flow manifold assembly comprises: a valve manifold defining at least one flow path; and an output manifold rotatably coupled to the valve manifold at the articulation joint and sealed to the valve manifold.

26. The electrosurgical device of clause 25, wherein the valve manifold comprises: a first port fluidically coupled to a first flow path; a second port fluidically coupled to a second flow path; a third port fluidically coupled to third flow path and to the first and the second flow paths, the third port fluidically coupled to the output manifold.

27. The electrosurgical device of clause 25, wherein the at least one flow path comprises at least one valve rotatably movable within the valve manifold and operatively coupled to at least one button supported by handle assembly.

28. The electrosurgical device of clause 27, wherein the at least one button comprises an arm portion defining a slot to engage a projecting tab on the at least one valve; and wherein linear motion of the arm portion translates to rotational motion of the valve to control flow through the valve.

29. The electrosurgical device of clause 24, wherein the locking mechanism comprises and articulation lock and spring, where in the articulation lock comprises a slot configured to engage a plurality of detents formed on the articulation joint and the spring biases the slot against detent to lock the articulation joint.

1. An electrosurgical device, comprising:
a handle assembly comprising:
a valve having an input port and an output port, the input port fluidically coupled to either an irrigation source or a suction source;
at least one button operatively coupled to the valve to control flow through the valve;
at least one switch to electrically couple energy from an energy source; and
a trigger;
a shaft comprising:
a slidable element operatively coupled to the trigger; and
an electrode electrically coupled to the switch;
wherein the trigger is operable to position the slidable element relative to the electrode to conceal or expose the electrode.

2. The electrosurgical device of claim 1, comprising a nozzle fluidically coupled to the output port of the valve, wherein the slidable element is fluidically coupled to the nozzle.

3. The electrosurgical device of claim 1, wherein the slidable element comprises a sheath fluidically coupled to the output port of the valve.

4. The electrosurgical device of claim 1, comprising a lever operatively coupled to the trigger and the slidable element such that the motion of the trigger is correlated to the motion of the slidable element.

5. The electrosurgical device of claim 4, wherein the slidable element is retracted in a proximal direction when the trigger is actuated in a proximal direction; and
wherein the slidable element is advanced in a distal direction when the trigger is actuated in a distal direction.

6. The electrosurgical device of claim 1, comprising a cam operatively coupled to the trigger and the slidable element such that the motion of the trigger is correlated to the motion of the slidable element.

7. The electrosurgical device of claim 1, comprising a gear assembly operatively coupled to the trigger and the slidable element such that the motion of the trigger is correlated to the motion of the slidable element.

8. The electrosurgical device of claim 1, comprising a cable pull system operatively coupled to the trigger and the slidable element such that the motion of the trigger is correlated to the motion of the slidable element.

9. The electrosurgical device of claim 1, comprising a spring operatively coupled to the trigger to return the trigger to its un-triggered state.

10. The electrosurgical device of claim 1, comprising a valve manifold to contain the valve.

11. The electrosurgical device of claim 1, wherein the button comprises a linear arm portion defining a slot and the valve comprises a projecting tab operatively coupled to the slot, wherein liner motion of the button arm translates into rotation of the valve.

12. The electrosurgical device of claim 1, wherein the valve is a stopcock valve.

13. The electrosurgical device of claim 1, comprising a locking mechanism operatively coupled to the trigger.

14. The electrosurgical device of claim 13, wherein the locking mechanism comprises a plurality of detents to lock the trigger at a plurality of positions.

15. The electrosurgical device of claim 13, wherein the trigger is movable from a first position to a second position and the locking mechanism maintains the trigger locked in the second position.

16. The electrosurgical device of claim 13, wherein the locking mechanism comprises a lever and a spring located on the trigger that automatically latches when the trigger is closed.

17. The electrosurgical device of claim 16, wherein the lever is pushed in an opposite direction from the latching direction to unlock the trigger.

18. The electrosurgical device of claim 16, comprising a spring biased member and a locking cam operatively coupled to the trigger, wherein the locking mechanism locks the trigger when the trigger moved in a first direction and unlocks the trigger when the trigger is moved again in the closed direction.

19. The electrosurgical device of claim 1, wherein the slide element is operatively coupled to a spring to return the slide mechanism to its starting position automatically.

20. An electrosurgical device, comprising:
a handle assembly defining a rigid wall;
a cam arm supported by the handle assembly and pivotally movable about a first pivot on one side and comprising a roller supported on another side, the cam arm defining a slot therebetween;
a first button supported by the handle assembly and pivotally movable about a second pivot, the first button comprising:
an arm; and
a projecting tab slidably engaged with the slot;
a second button pivotally coupled to the cam arm at the first pivot; and
a bias element acting on the arm of the first button to force the cam arm to pivotally move in a first direction and the roller to move toward the rigid wall of the handle assembly;
wherein pressing the first button overcomes the force of the bias element and the projecting tab applies a force on the slot to pivotally move the cam arm in a second direction and to move the roller away from the rigid wall of the handle assembly.

21. The electrosurgical device of claim 20, comprising a flexible tube located between the rigid wall and the roller such that the roller applies a pinching force against the tube when the cam arm is pivotally forced in the first direction by the bias element.

22. The electrosurgical device of claim 21, wherein the pinching force against the tube is removed when the cam arm is pivotally moved in the second direction.

23. The electrosurgical device of claim 20, wherein the second button is operable to partially control the force applied to the cam arm.

24. An electrosurgical device comprising:
a handle assembly comprising an articulating handle comprising a proximal housing and a distal housing rotatably coupled at an articulation joint;
a sealed fluid flow manifold assembly configured to articulate about the articulation joint; and
a locking mechanism positioned at the articulation joint to lock the proximal housing and the distal hosing in a configuration.

25. The electrosurgical device of claim 24, wherein the articulating fluid flow manifold assembly comprises:
a valve manifold defining at least one flow path; and
an output manifold rotatably coupled to the valve manifold at the articulation joint and sealed to the valve manifold.

26. The electrosurgical device of claim 25, wherein the valve manifold comprises:
a first port fluidically coupled to a first flow path;
a second port fluidically coupled to a second flow path;
a third port fluidically coupled to third flow path and to the first and the second flow paths, the third port fluidically coupled to the output manifold.

27. The electrosurgical device of claim 25, wherein the at least one flow path comprises at least one valve rotatably movable within the valve manifold and operatively coupled to at least one button supported by handle assembly.

28. The electrosurgical device of claim 27, wherein the at least one button comprises an arm portion defining a slot to engage a projecting tab on the at least one valve; and
wherein linear motion of the arm portion translates to rotational motion of the valve to control flow through the valve.

29. The electrosurgical device of claim 24, wherein the locking mechanism comprises and articulation lock and spring, where in the articulation lock comprises a slot configured to engage a plurality of detents formed on the articulation joint and the spring biases the slot against detent to lock the articulation joint.
